(19) United States
(12) Patent Application Publication
(10) Pub. No.: US 2024/0398906 A1
(43) Pub. Date: Dec. 5, 2024

(54) PROPEPTIDE OF LYSYL OXIDASE FOR TREATMENT OF CANCER

(71) Applicants: Yeda Research and Development Co. Ltd., Rehovot (IL); Ramot at Tel-Aviv University Ltd., Tel-Aviv (IL)

(72) Inventors: Irit SAGI, Rehovot (IL); Nikolaos A. AFRATIS, Rehovot (IL); Inna SOLOMONOV, Rehovot (IL); Carmit LEVY, Tel Aviv (IL); Shivang PARIKH, Tel Aviv (IL)

(73) Assignees: Yeda Research and Development Co. Ltd., Rehovot (IL); Ramot at Tel-Aviv University Ltd., Tel-Aviv (IL)

(21) Appl. No.: 18/804,150

(22) Filed: Aug. 14, 2024

Related U.S. Application Data
(63) Continuation of application No. PCT/IL2023/050163, filed on Feb. 16, 2023.
(60) Provisional application No. 63/311,047, filed on Feb. 17, 2022.
(60) Provisional application No. 63/356,541, filed on Jun. 29, 2022.

(51) Int. Cl. A61K 38/44; A61K 38/17; A61K 45/06; A61P 35/00

(52) U.S. Cl. CPC A61K 38/44; A61K 38/1709; A61K 45/06; A61P 35/00; C12Y 104/03013

(57) ABSTRACT

A method of treating a cancer which is characterized by an up-regulation of expression of lysyl oxidase (LOX) and heat shock protein 70 (HSP70) is disclosed. The method comprises administering to the subject a therapeutically effective amount of a polypeptide comprising a propeptide of lysyl oxidase (LOX), said polypeptide being devoid of LOX catalytic activity, wherein said polypeptide binds to both LOX and heat shock protein 70 (HSP70) with a EC₅₀ of less than 100 nM.

### RELATED APPLICATIONS

This application is a Continuation of PCT Patent Application No. PCT/IL2023/050163 having International filing date of Feb. 16, 2023, which claims the benefit of priority under 35 USC § 119 (e) of U.S. Provisional Patent Application Nos. 63/311,047 filed on Feb. 17, 2022 and 63/356,541 filed on Jun. 29, 2022. The contents of the above applications are all incorporated by reference as if fully set forth herein in their entirety.

### SEQUENCE LISTING STATEMENT

The XML file, entitled 100819SequenceListing.xml, created on Jul. 2, 2024, comprising 23,662 bytes, submitted concurrently with the filing of this application is incorporated herein by reference.

### FIELD AND BACKGROUND OF THE INVENTION

The present invention, in some embodiments thereof, relates to methods of treating cancer which are characterized by an up-regulation in the extracellular matrix molecules lysyl oxidase (LOX) and heat-shock protein 70 (HSP70).

Malignant melanoma is the most aggressive and treatment-resistant human skin cancer. In the early 2000s, chemotherapy was the only therapeutic approach with median survival of 6-9 months. Later on, the first targeted therapy with vemurafenib for BRAF-V600 mutant melanoma patients was approved. Even though BRAF-V600 therapies showed a strong activity for patients with this mutation, their efficacy was limited because of adverse events and resistance mechanisms (e.g. Giunta et al., 2020, Ther Adv Med Oncol 12, 1758835920925219). Metastatic melanoma has been characterized as an immunologically affected tumor and the recent approval of immune checkpoint inhibitor drugs (such as ipilimumab, nivolumab and pembrolizumab), as well as targeted therapies lead to 50% survival 5 years after diagnosis (e.g. Larkin et al., 2019, N Engl J Med 381, 1535-1546). However, 50% of the patients still do not respond to existing therapies (Giunta et al., 2020, ibid). For this purpose, investigation of new biomarkers that could be used to differentiate between the patients will lead to the development of new therapeutic approaches that combines immunotherapeutic strategies and targeted therapies.

Interactions of tumor cells with the surrounding tissue are regulated by cell-ECM interactions (Afratis et al. Adv Drug Deliv Rev 129, 4-15) during tumor progression. In many cancer types, including melanoma, expression of ECM components, such as collagen I and III, and ECM-modifying enzymes, such as lysyl oxidases (LOX) and LOX-like proteins (LOXL), is altered and lead to increase of matrix stiffness (e.g. Kai et al., 2019, Dev Cell 49, 332-346). Modified-ECM formats a physical barrier and prevents sufficient T-cells movement as well as the T-cell infiltration and impairs immune surveillance of tumors. Considering the importance of ECM, among the novel biomarkers whose inhibition could lead to elimination of advanced melanoma are the ECM molecules, lysyl oxidase (LOX) and heat-shock protein 70 (HSP70). Both molecules are overexpressed in various cancer types, such as breast, colon and gastric cancers.

It has been reported that targeting LOX improves the efficacy of chemotherapy in different kind of solid tumors (Le Calve et al., 2016, Oncotarget 7, 32100-32112). LOX is overexpressed in melanoma cells and its inhibition by β-aminopropionitrile (BAPN) suppresses invasive growth of melanoma cells. However, previous studies reported high toxicity in long term clinical use (Piersma et al., 2020, Biochim Biophys Acta Rev Cancer 1873, 188356). Recently, another small molecule LOX inhibitor has been tested in Phase I clinical trial for other indications (myelofibrosis and pancreatic cancer) (Hauge and Rofstad, 2020, J Transl Med 18, 207). Activation mechanism of LOX is mediated via BMP-1, whose proteolytic activity cleaves the LOX pro-peptide (LOX-PP) and release the active form of LOX on ECM milieu. Interestingly, the endogenous LOX inhibitor, LOX-PP, is reported to have tumor-suppressor function via blocking LOX activity (Ozdener et al., 2016, Mol Oncol 10, 1-23).

Elevated expression of the inducible HSP70 is known to be correlated with poor prognosis in many cancer types. HSP70 family members have been implicated in metastasis formation as well (e.g. Garg et al., 2010 Cancer 116, 3785-3796). Elevated HSP70 expression correlates with lymph node metastases and decreased survival in breast cancer models (Kluger et al., 2005, Cancer Res 65, 5578-5587). Notably, HSP70 has been shown to enhance invasion and migration in several cancer types, including lymph node metastasis in breast cancer, gastric cancers, cervical and bladder cancer (e.g. Budina-Kolomets et al., 2016, Cancer Res 76, 2720-2730 Kluger et al., 2005, ibid). Interestingly, HSP70 inhibition has been successfully used in cellular and in vivo melanoma model, especially as an adjuvant approach for overcoming the resistance to BRAF inhibitors, which is frequently observed in melanoma patients (Budina-Kolomets et al., 2016, ibid). Mechanistically, the carboxyl-terminus of CHIP downregulates Met, one of the key receptors that triggers the epithelial to mesenchymal transition (EMT) via a switch from HSP70 chaperone activity to proteasomal targeting. HSP70, in TGF-beta-induced EMT, blocks TGF-beta signaling by impeding Smad2 phosphorylation.

Additional background art includes WO2020/222241.

### SUMMARY OF THE INVENTION

According to an aspect of the present invention there is provided a method of treating a cancer which is characterized by an up-regulation of expression of lysyl oxidase (LOX) and heat shock protein 70 (HSP70), the method comprising administering to the subject a therapeutically effective amount of a polypeptide comprising a propeptide of lysyl oxidase (LOX), the polypeptide being devoid of LOX catalytic activity, wherein the polypeptide binds to both LOX and heat shock protein 70 (HSP70) with a EC₅₀ of less than 100 nM, thereby treating the cancer.

According to another aspect of the present invention there is provided a polypeptide comprising a propeptide of lysyl oxidase (LOX), the polypeptide being devoid of LOX catalytic activity for use in treating a cancer which is characterized by an up-regulation of expression of lysyl oxidase (LOX) and heat shock protein 70 (HSP70) in a subject in need thereof.

According to an embodiment of the present invention, the cancer is selected from the group consisting of melanoma, prostate adenocarcinoma, testis embryonal carcinoma, ovarian cancer, uterus carcinoma, pancreatic adenocarcinoma, astrocytoma and glioblastoma.

According to an embodiment of the present invention, the cancer is melanoma.

According to an embodiment of the present invention, the cancer is metastasized.

According to an embodiment of the present invention, the method further comprises selecting the subject for treatment by analyzing the level of the LOX and the HSP70 in a sample of the subject, wherein when the level is above a predetermined level, the subject is selected as a candidate for treatment.

According to another aspect of the present invention there is provided a article of manufacture comprising:
(i) a polypeptide comprising a propeptide of lysyl oxidase (LOX), the polypeptide being devoid of LOX catalytic activity, wherein the polypeptide binds to both LOX and heat shock protein 70 (HSP70) with a EC₅₀ of less than 100 nM; and
(ii) an immune modulating agent.

According to an embodiment of the present invention, the polypeptide and the immune modulating agent are formulated in a single pharmaceutical composition.

According to an embodiment of the present invention, the polypeptide and the immune modulating agent are formulated in separate pharmaceutical compositions.

According to an embodiment of the present invention, the polypeptide comprises the amino acid sequence as set forth in SEQ ID NO: 1.

According to an embodiment of the present invention, the polypeptide is glycosylated on at least one of N81, N97 and N144 of SEQ ID NO: 1.

According to an embodiment of the present invention, the polypeptide is glycosylated on at least two of N81, N97 and N144 of SEQ ID NO: 1.

According to an embodiment of the present invention, the polypeptide is glycosylated on N81, N97 and N144 of SEQ ID NO: 1.

According to an embodiment of the present invention, the polypeptide is not glycosylated on N81, N97 and N144 of SEQ ID NO: 1.

According to an embodiment of the present invention, the propeptide of LOX is of human LOX.

According to an embodiment of the present invention, the polypeptide comprising a modification which imparts the polypeptide with enhanced stability under physiological conditions as compared to a native form of the polypeptide not comprising the modification.

According to an embodiment of the present invention, the modification comprises a proteinaceous modification.

According to an embodiment of the present invention, the proteinaceous modification is selected from the group consisting of immunoglobulin, human serum albumin, and transferrin.

According to an embodiment of the present invention, the immunoglobulin comprises an Fc domain.

According to an embodiment of the present invention, the polypeptide comprises the amino acid sequence as set forth in SEQ ID NOs: 12 or 13.

According to an embodiment of the present invention, the polypeptide is a chimeric polypeptide.

According to an embodiment of the present invention, the modification comprises a chemical modification.

According to an embodiment of the present invention, the chemical modification is a polymer.

According to an embodiment of the present invention, the polymer is selected from the group consisting of a polycationic polymer, a non-ionic water-soluble polymer, a polyether polymer and a biocompatible polymer.

According to an embodiment of the present invention, the method further comprises administering to the subject an immune modulating agent.

According to an embodiment of the present invention, the immune modulating agent comprises an immune checkpoint inhibitor.

According to an embodiment of the present invention, the immune checkpoint inhibitor is an anti-PD-1 antibody.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fec.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-H: LOX and HSP70 are biomarkers in malignant melanoma and are related to poor survival.

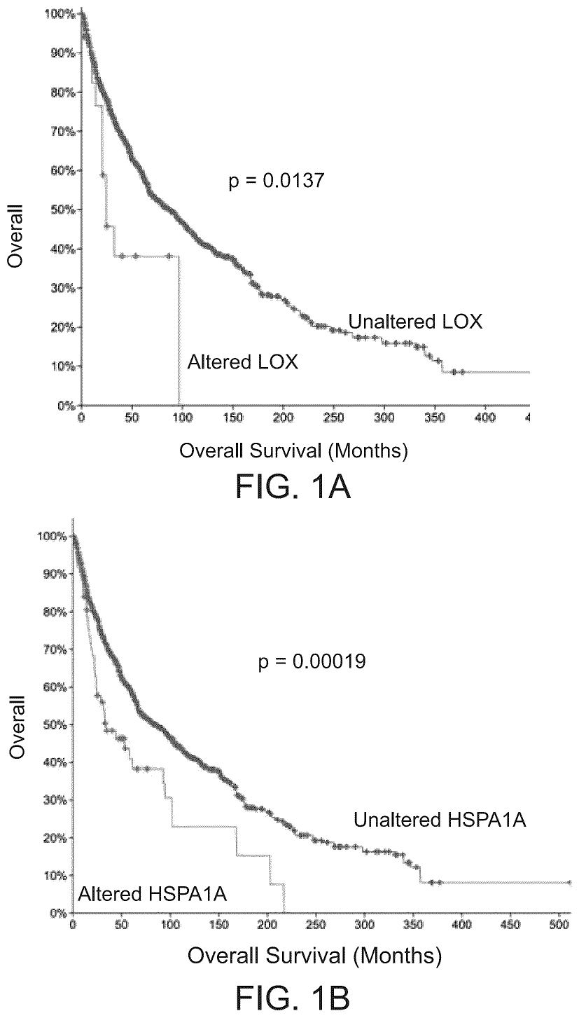

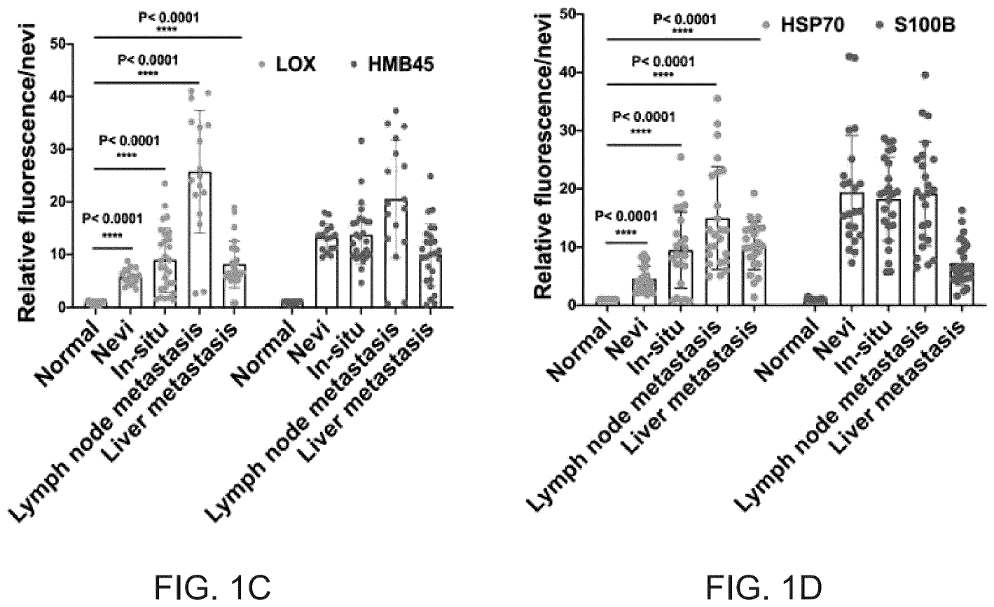

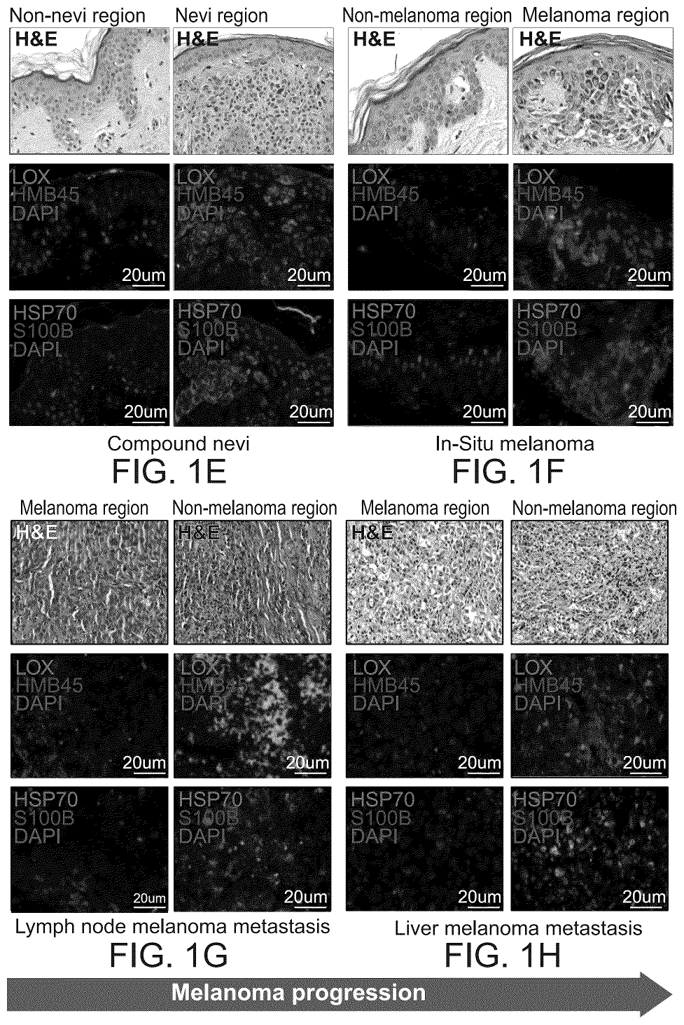

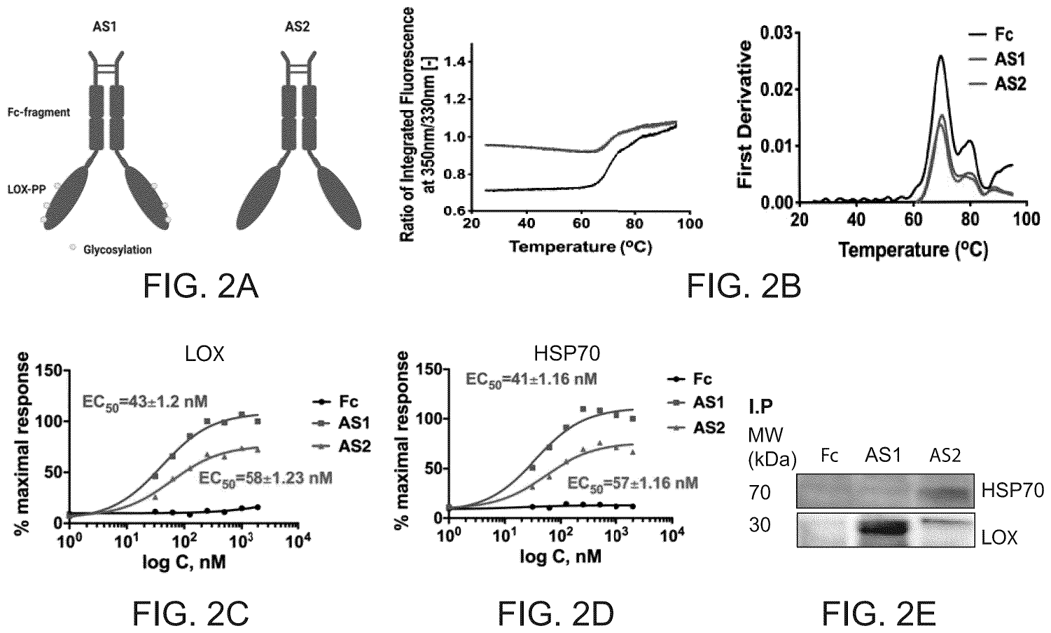

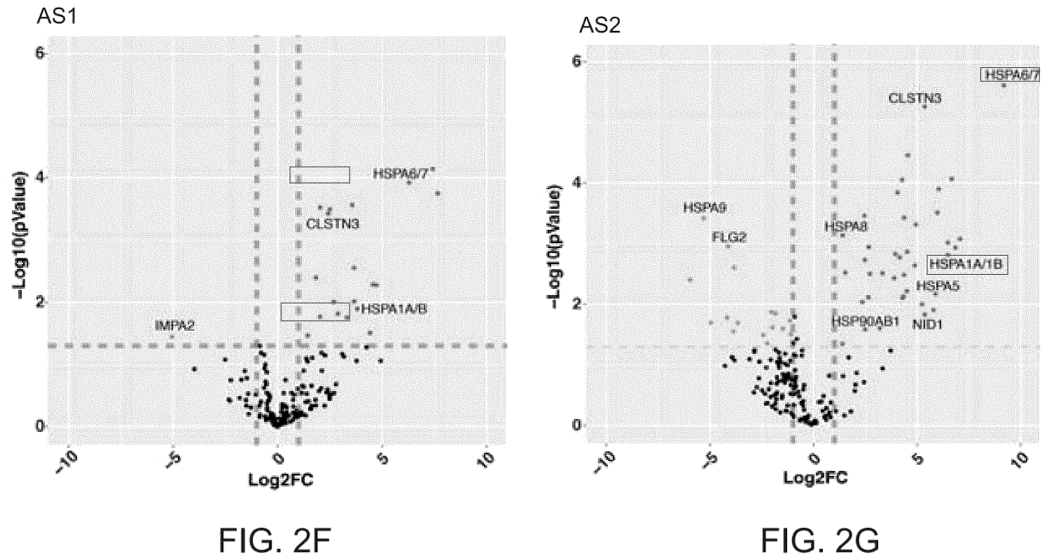

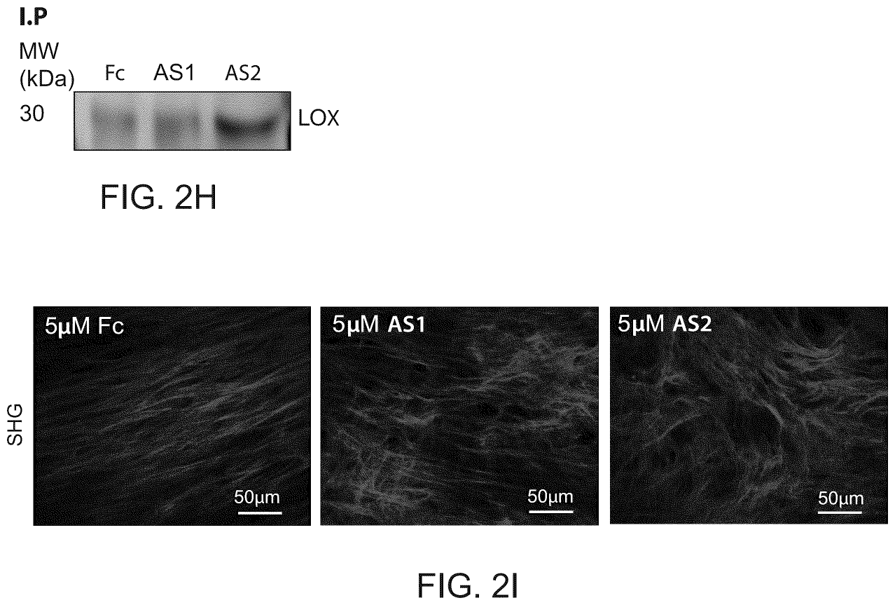

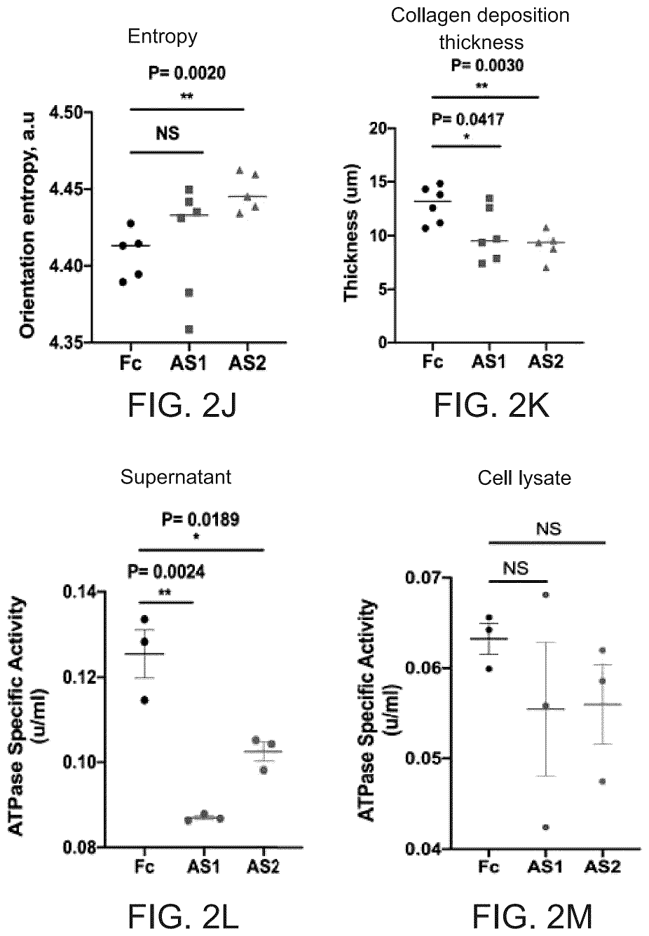

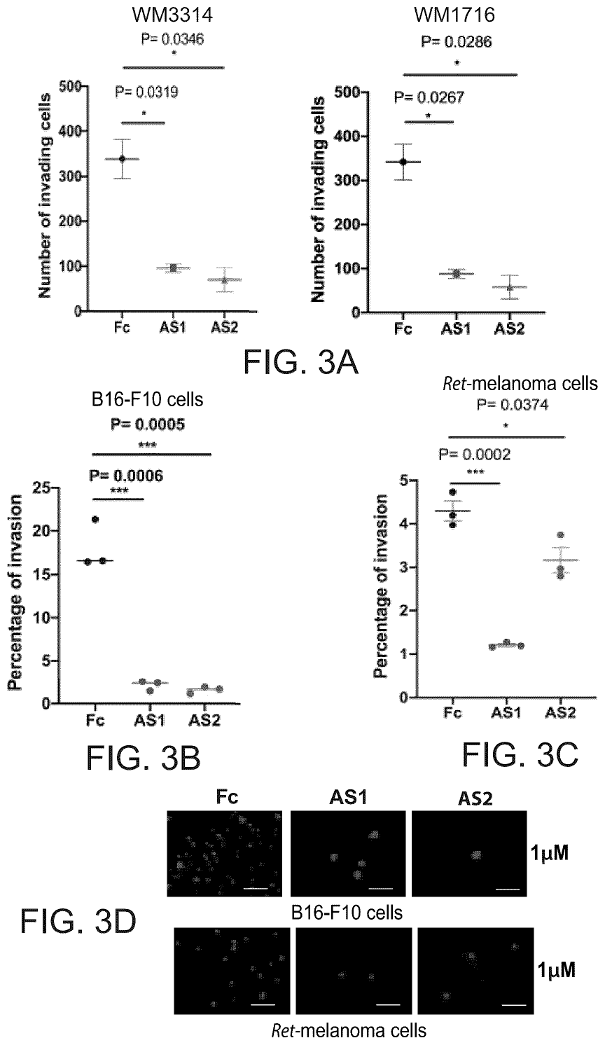

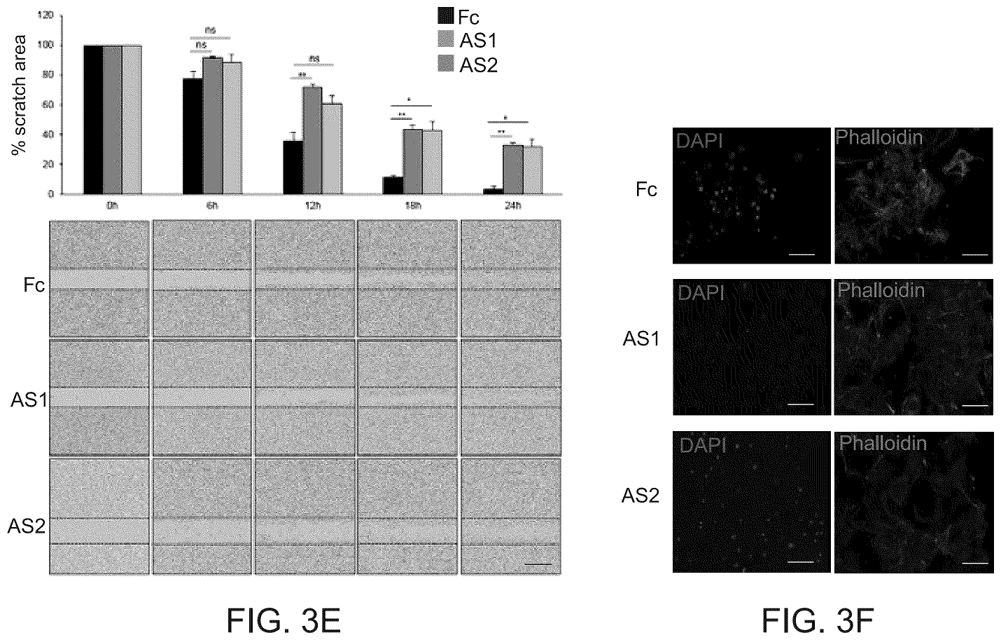

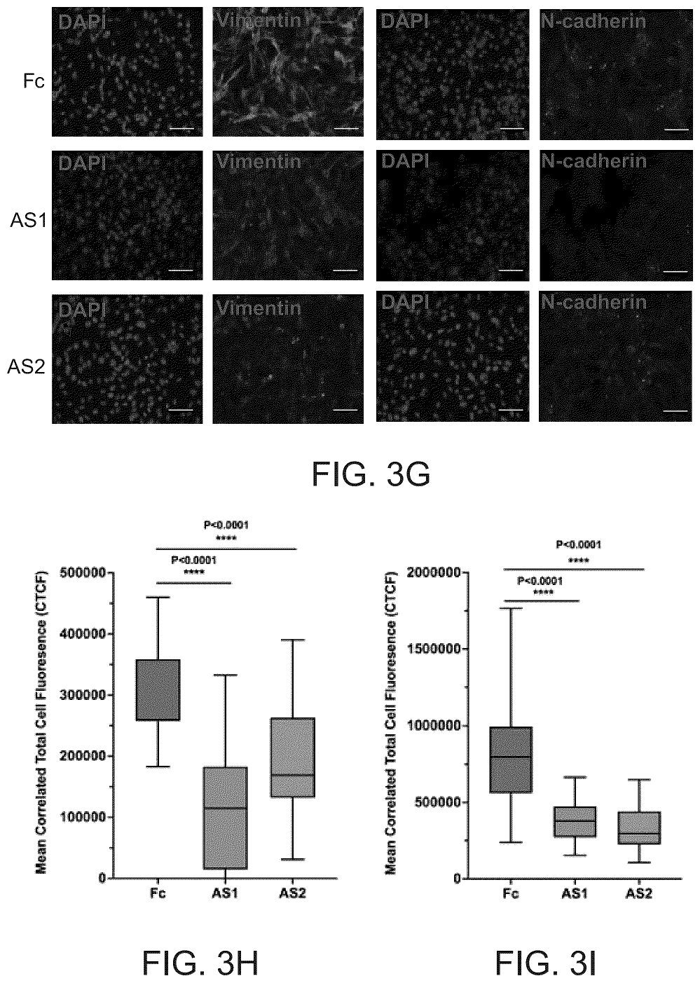

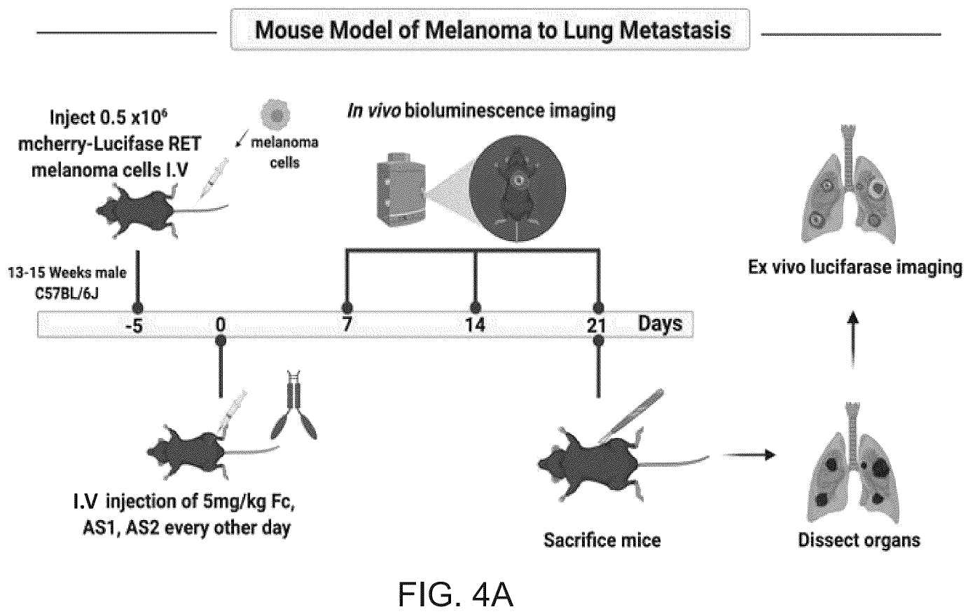

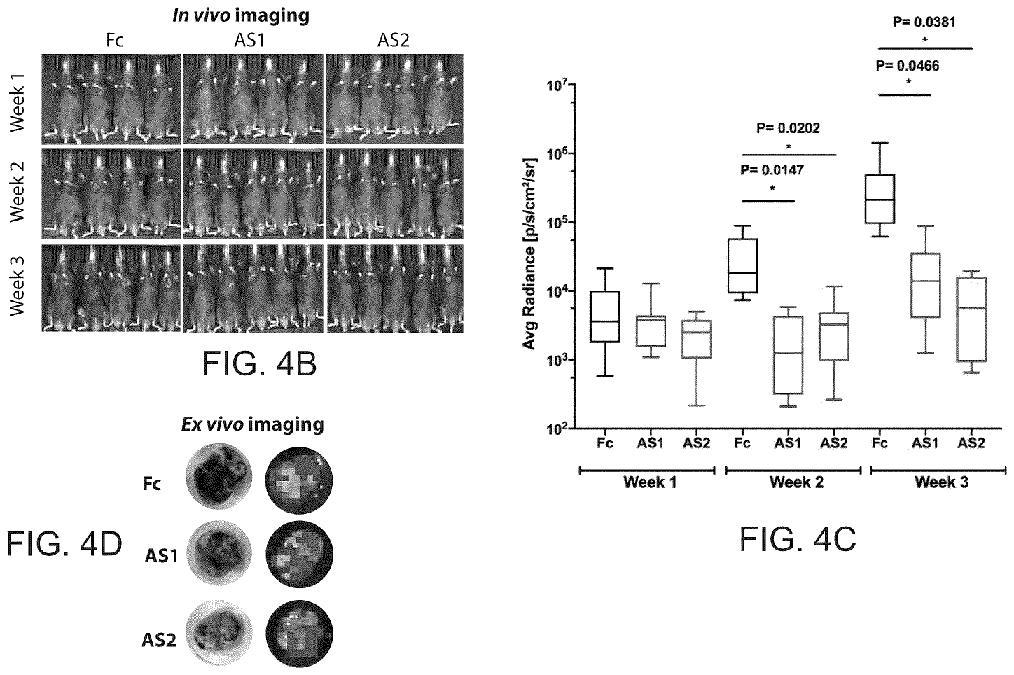

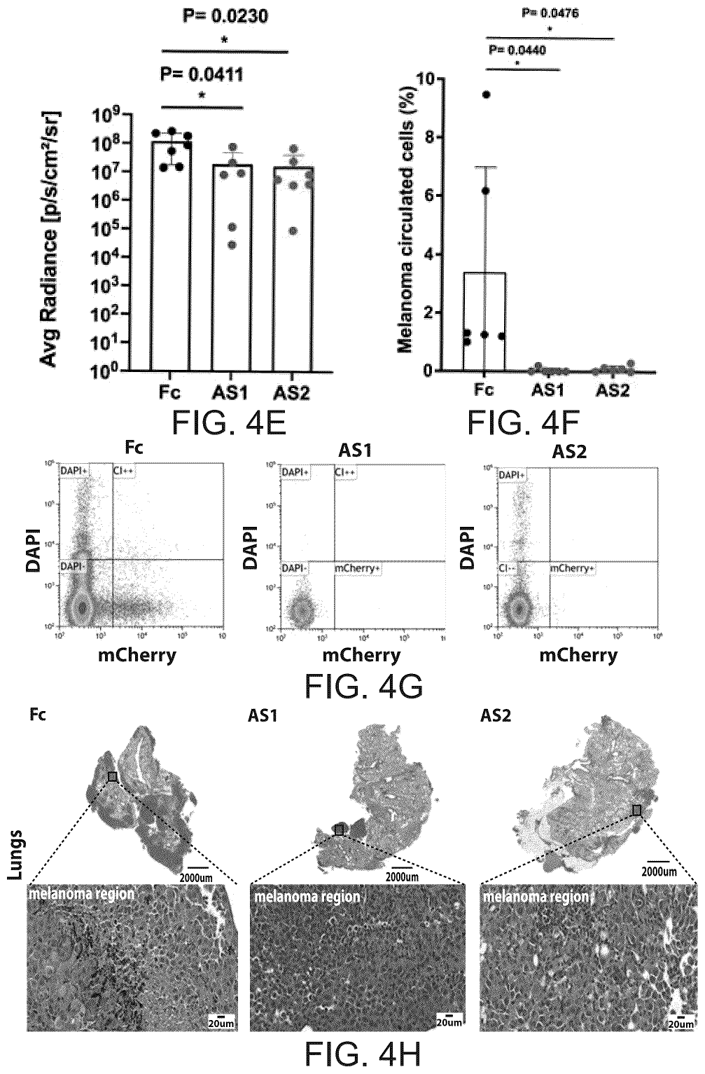

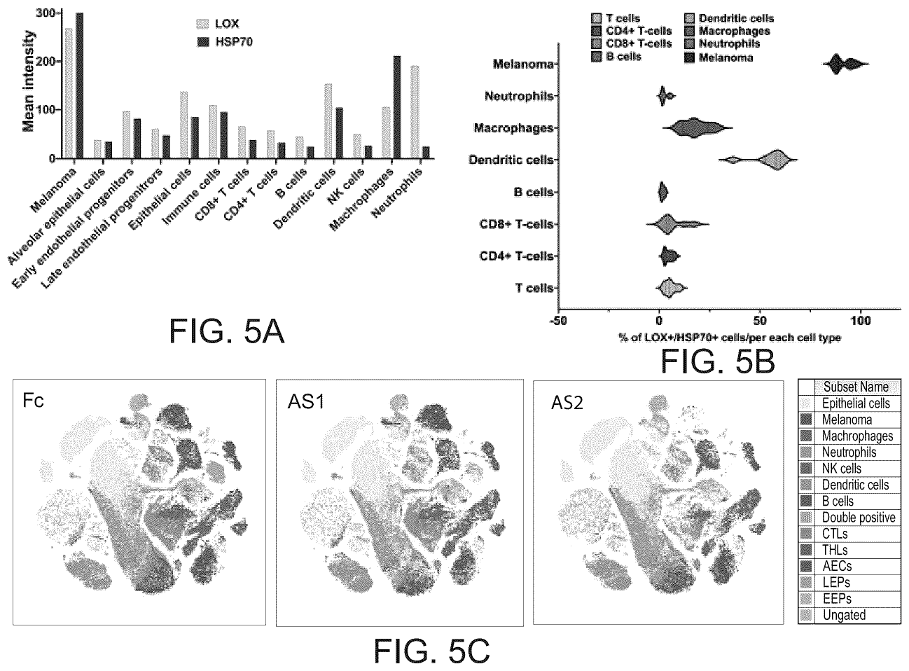

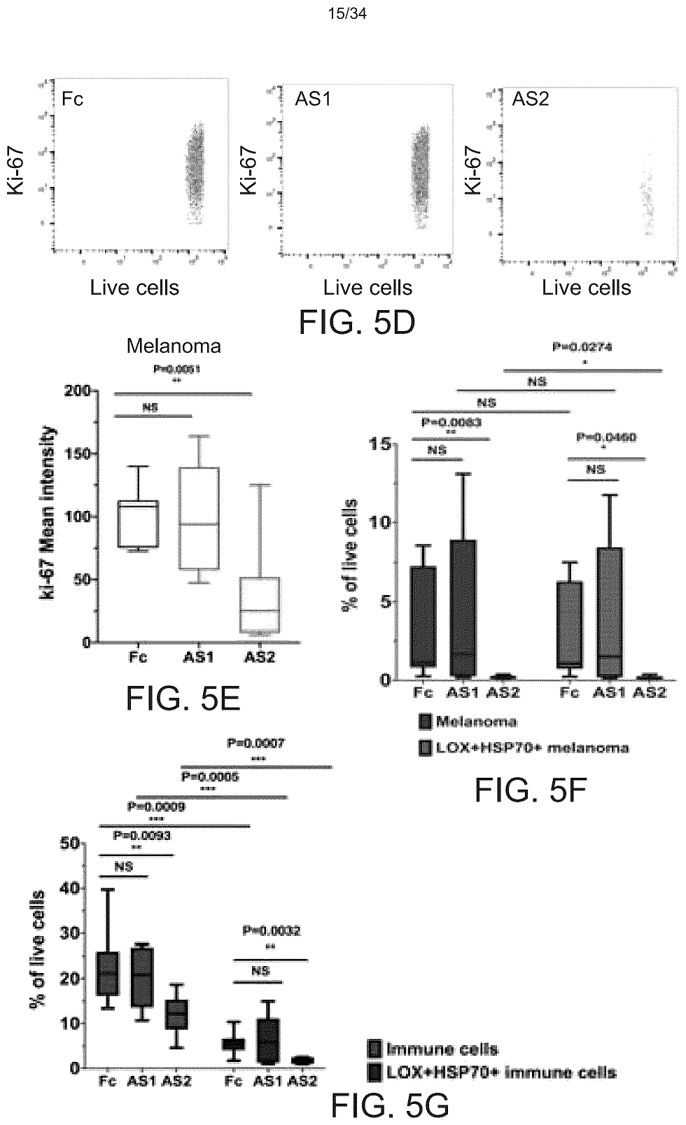

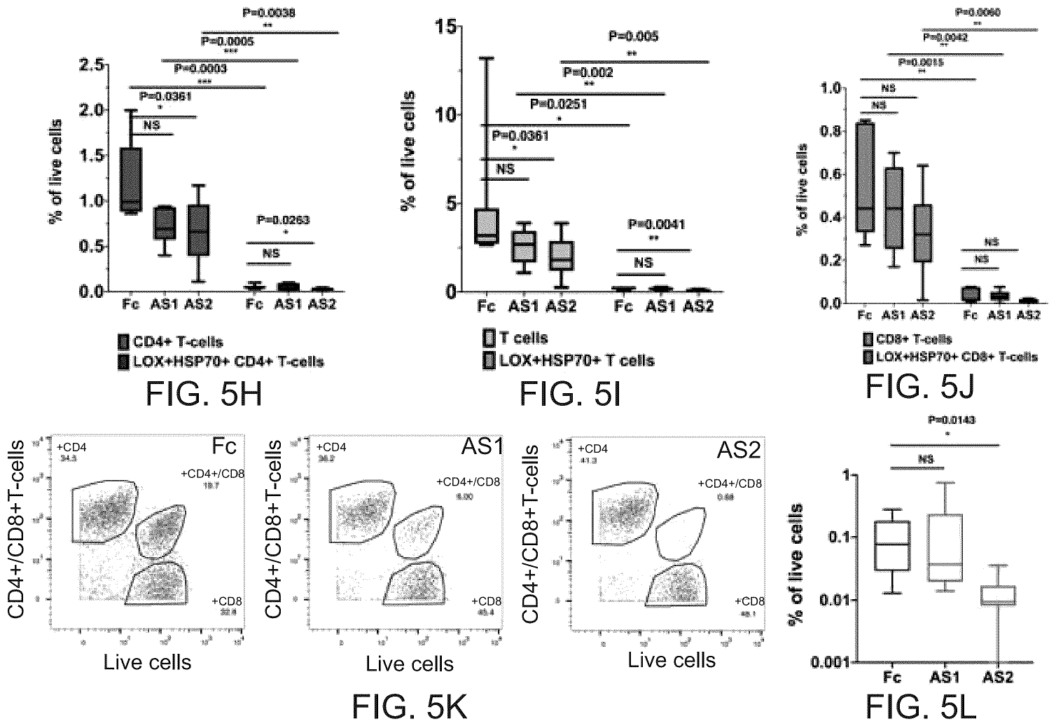

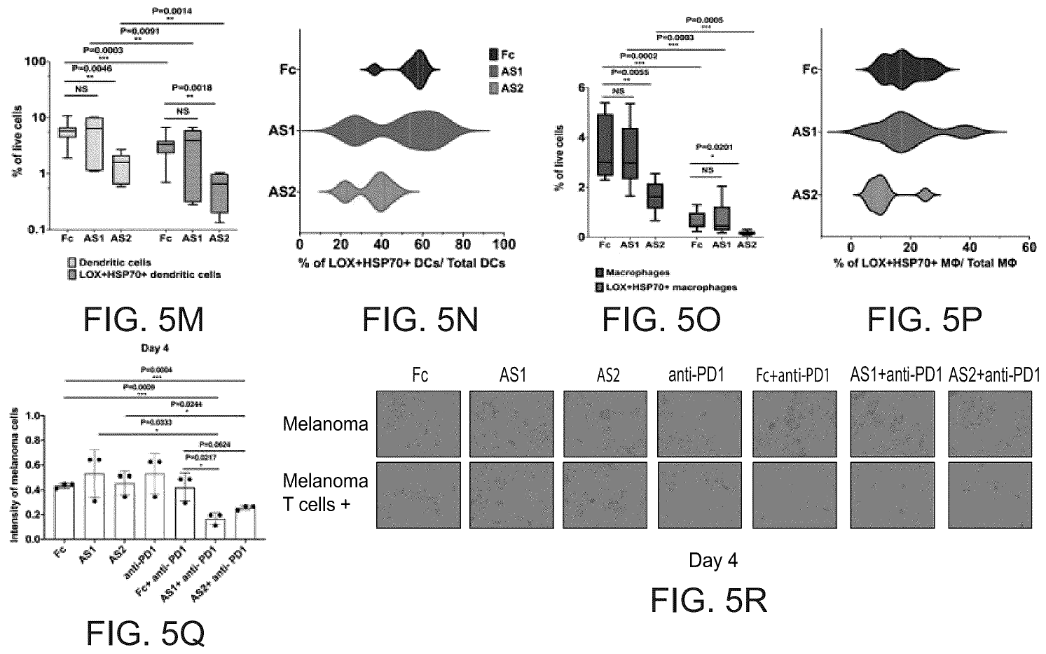

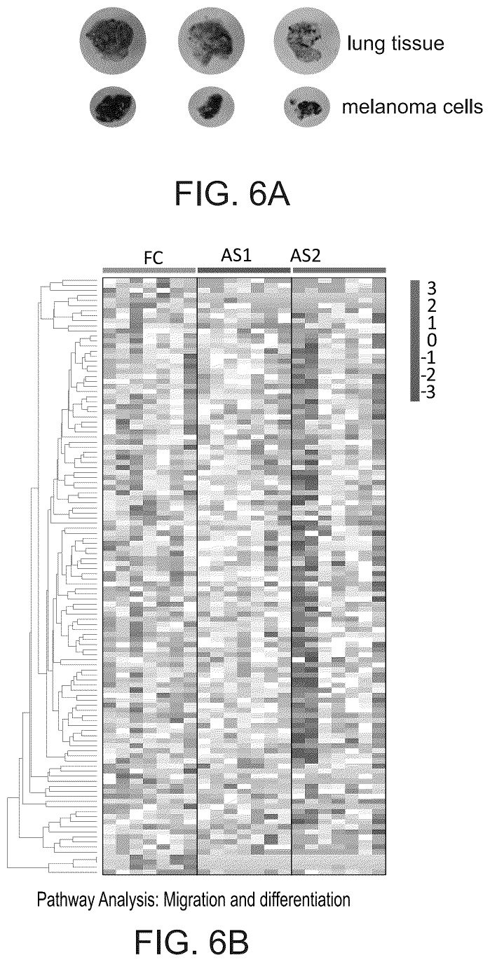

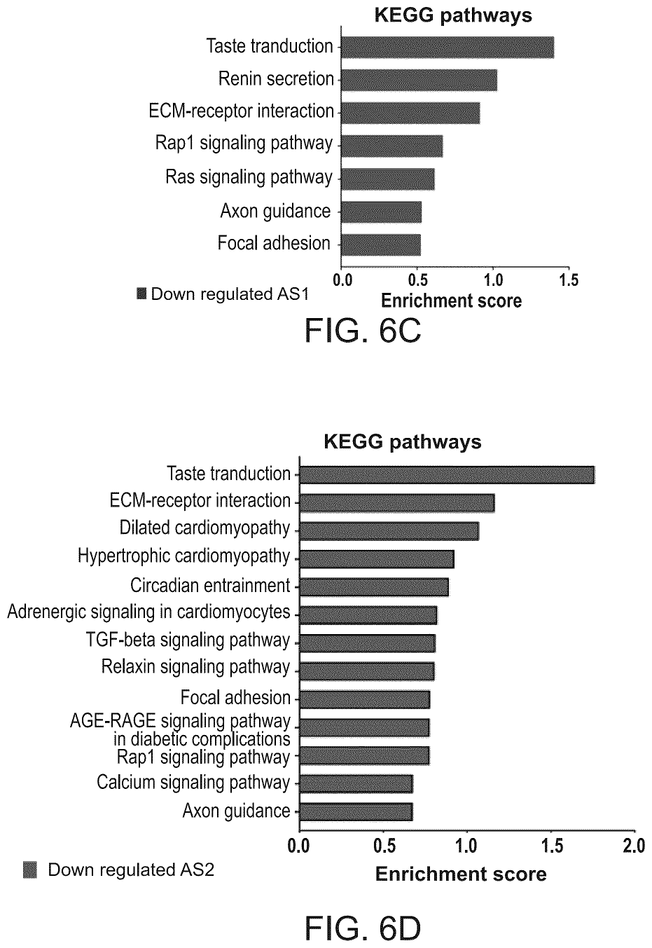

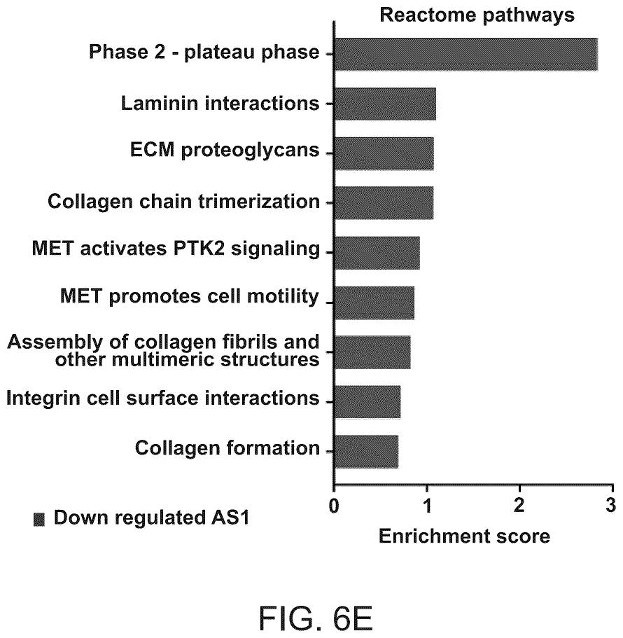

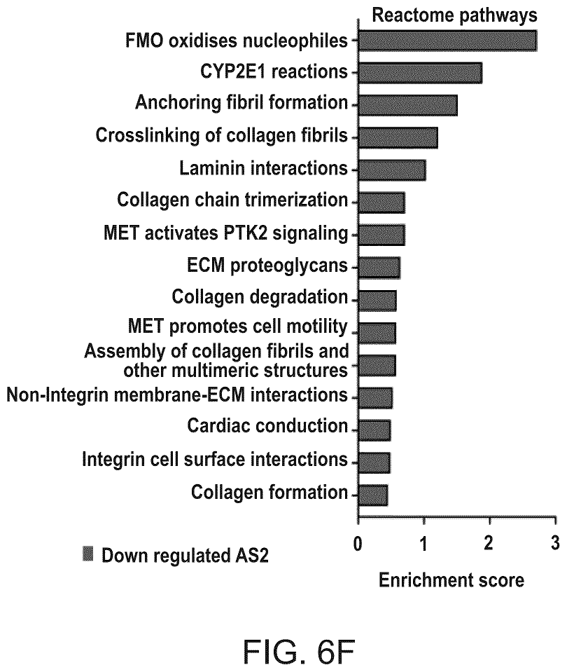

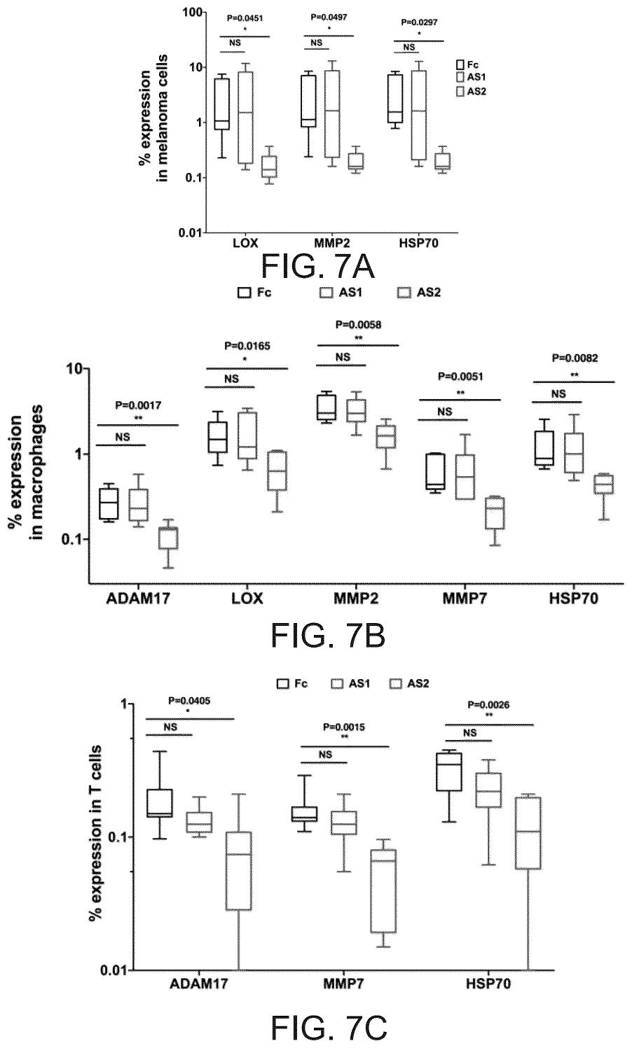

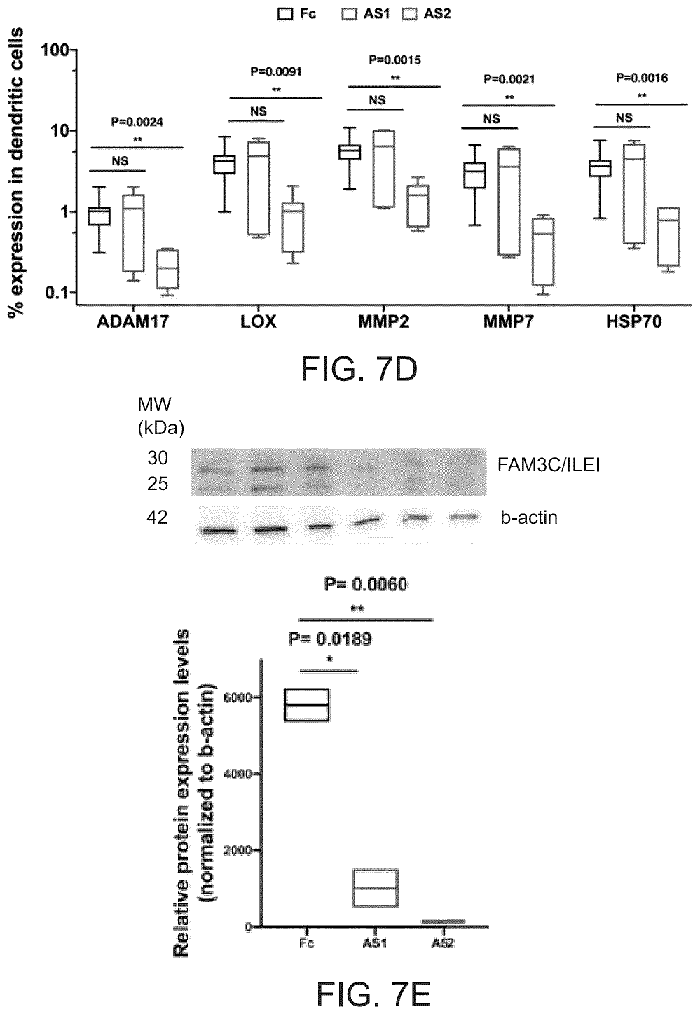

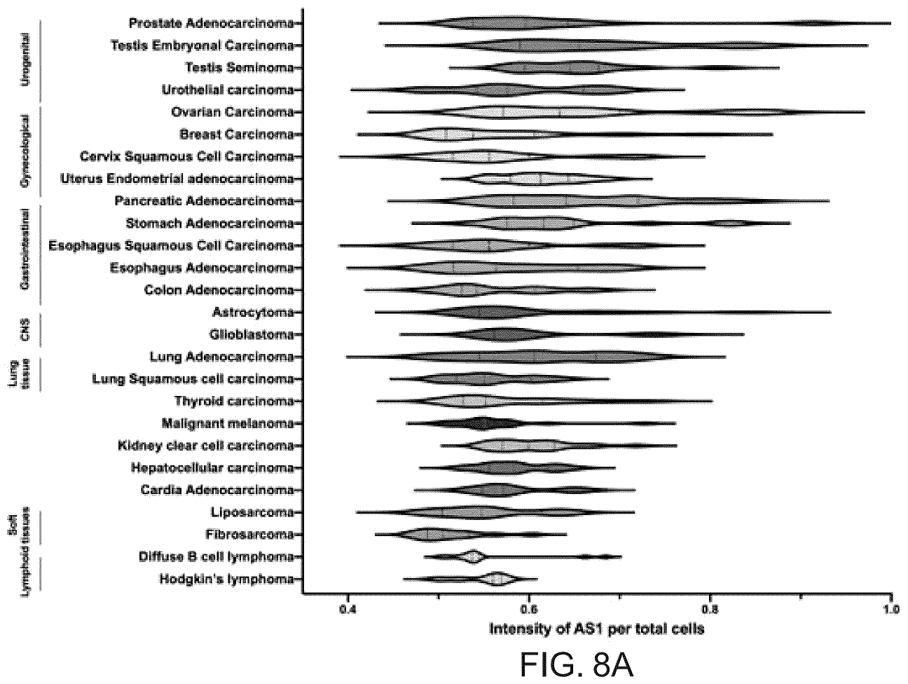

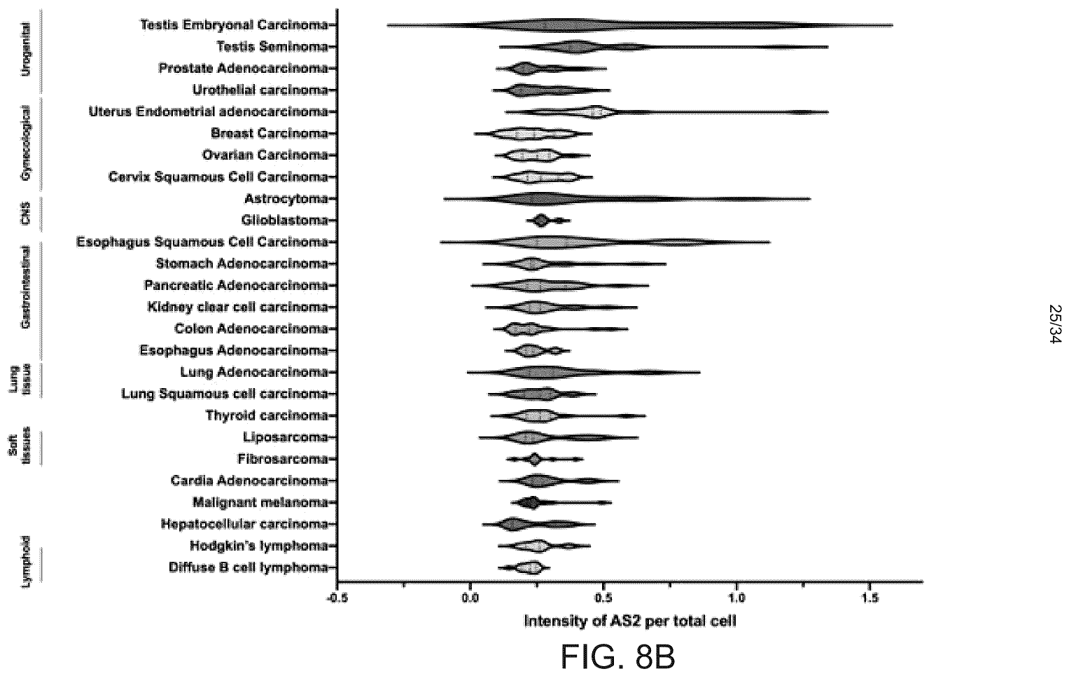

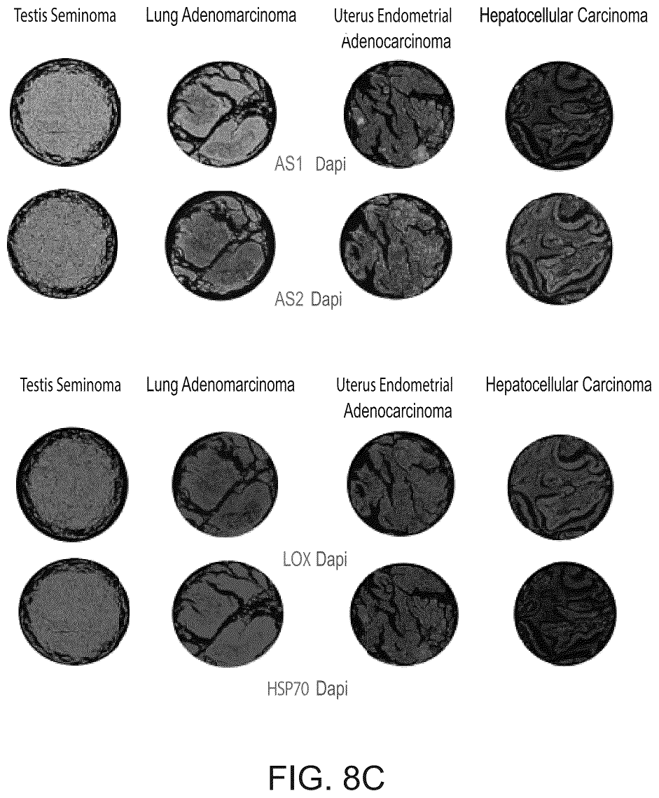

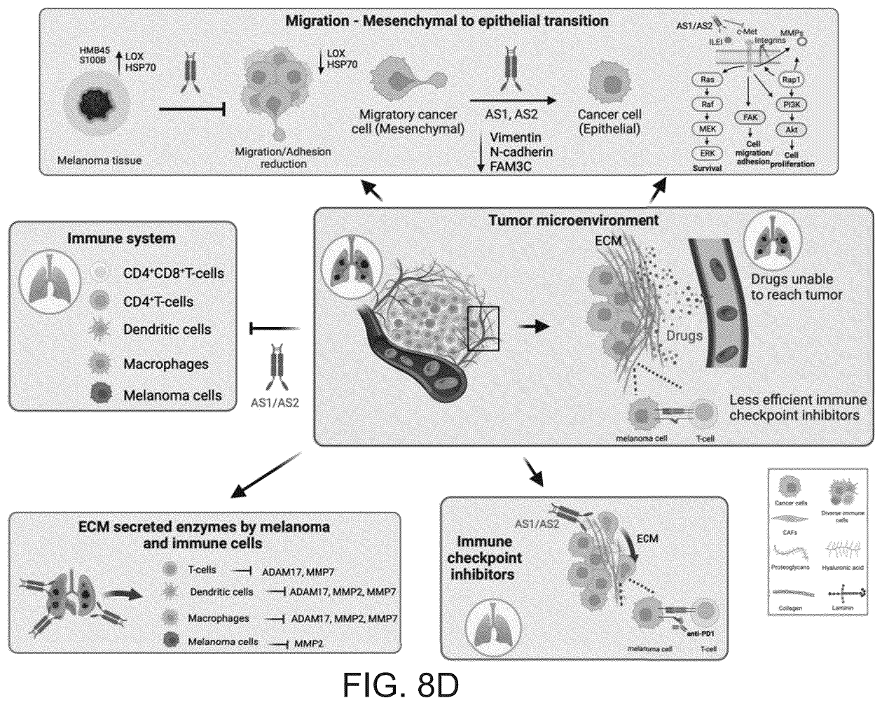

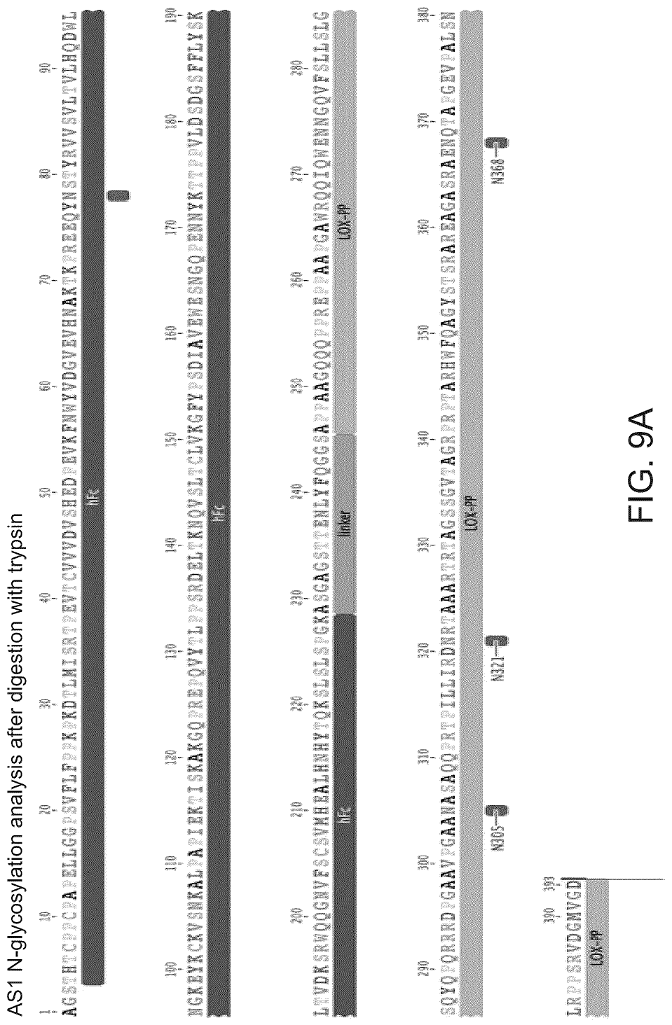

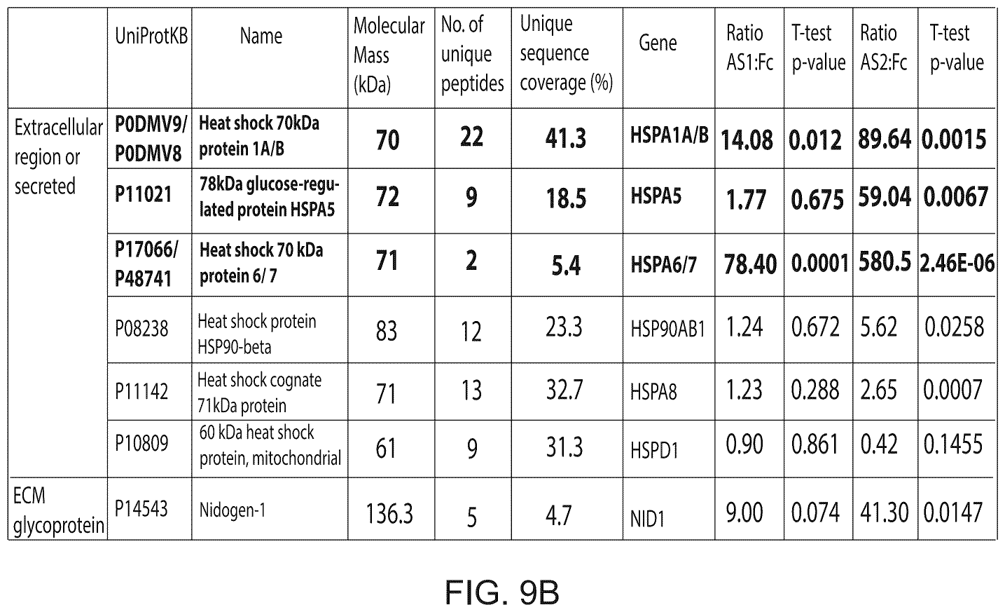

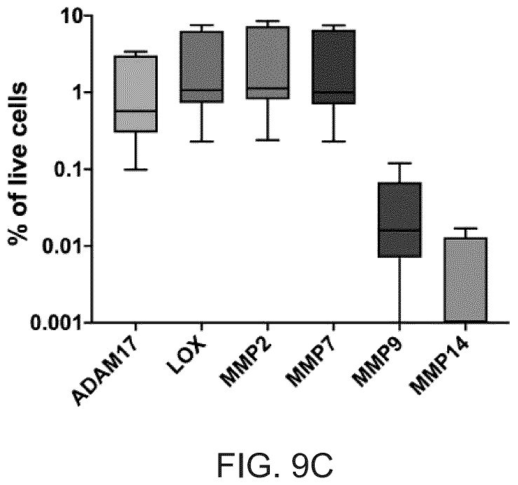

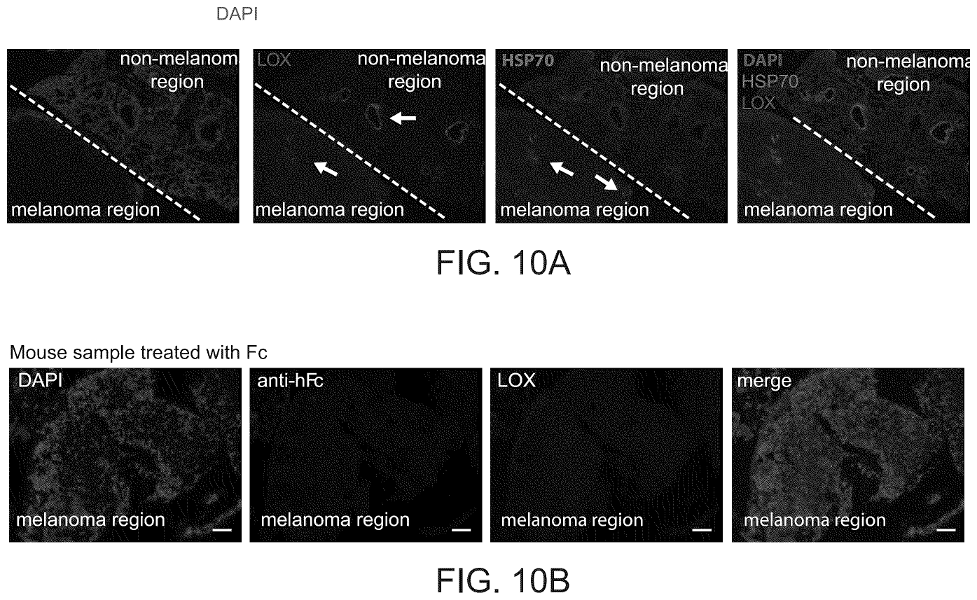

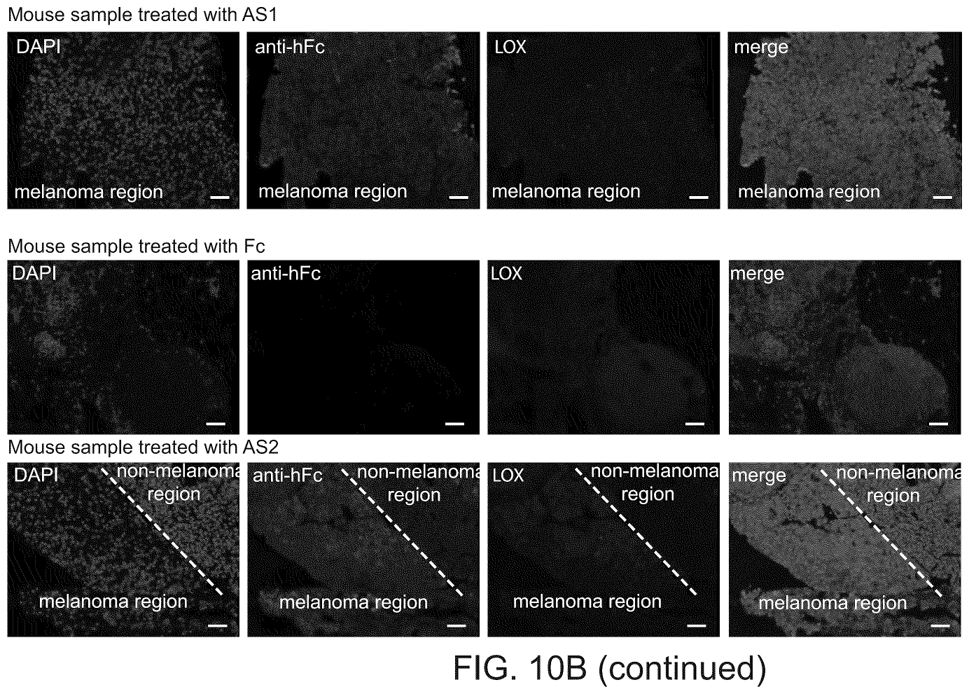

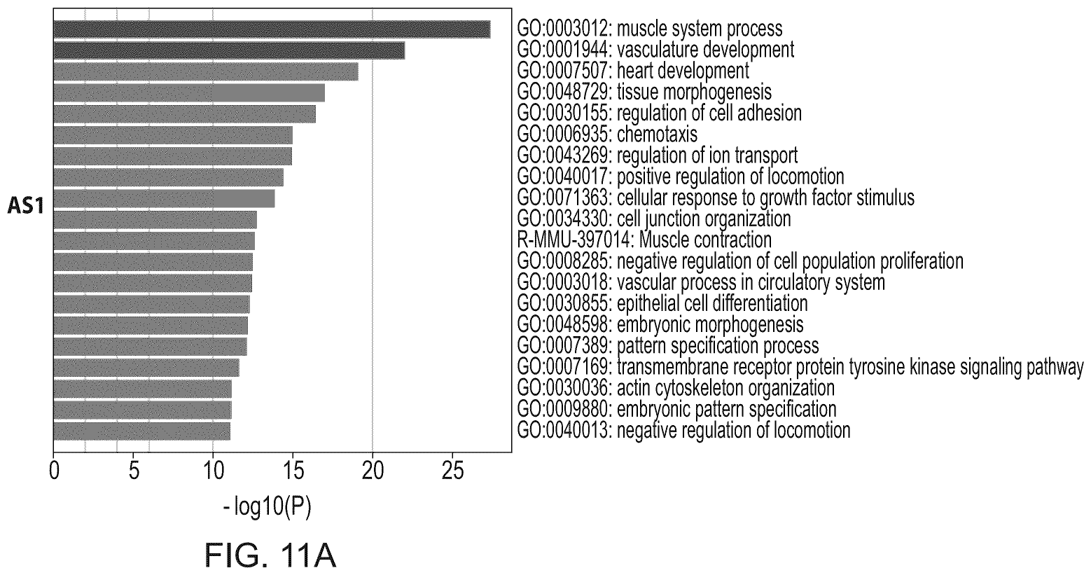

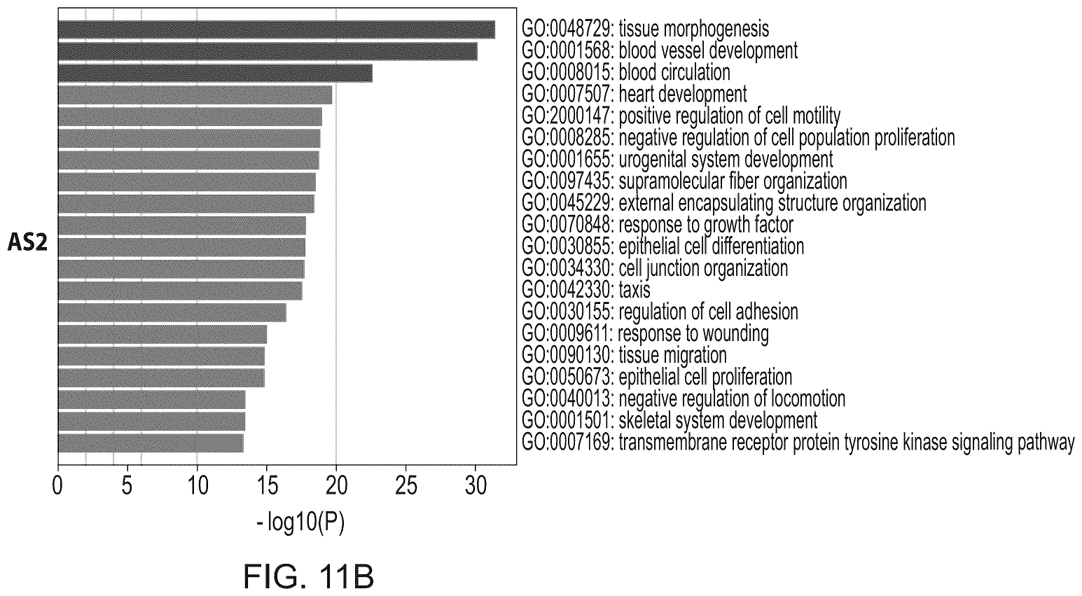

A, Kaplan-Meier graphs describing the probability of overall survival of melanoma patients with altered LOX levels (17) compared to those with unaltered level (1147), Median Months Overall (95% CI): altered group 24.53 (20.35-NA) and unaltered group 61.10 (51.90-66.67) (Database: cbioportal (dot) org). B, Kaplan-Meier graph for of melanoma patients with altered HSP70 (HSPAIA) levels (62) compared to those with unaltered level (1390). Median Months Overall (95% CI): altered group 34.75 (23.92-94.60) and unaltered group 65.44 (60.18-71.75). (Database: cbioportaldotorg). C, Quantification of LOX and HMB45 (a melanoma marker) levels evaluated by immunostaining in tissue sections. Relative fluorescence intensity levels of human melanoma tissues from FIGS. 1E-H were quantified and compared to normal specimens. D, Quantification of HSP70 and S100B (a melanoma marker) expression. Relative fluorescence intensity levels of melanoma tissues from FIGS. 1E-H were quantified and compared to normal specimens. Human biopsies were stained with Hematoxylin & Eosin (upper panel) and by immunofluorescence with antibodies directed against LOX and HMB45 (middle panel) and against HSP70 and S100B (lower panel) in E, nevi and non-nevi regions. F, melanoma and non-melanoma regions. G, in melanoma and non-melanoma regions of lymph node tissues from metastatic melanoma patients. H, melanoma and non-melanoma regions of liver tissues specimens from melanoma to liver metastasis patients. Data are presented as mean±SEM; values of p<0.05 were considered statistically significant (*p<0.05, **p<0.01, ***p<0.001).

FIGS. 2A-M: Design and evaluation of the biological activity AS1 and AS2 inhibitors.

A, Schematic representation of fused LOX-PP and IgG1 Fc antibody fragment creating an antibody-like structure of glycosylated (AS1) and non-glycosylated (AS2) inhibitors. B, Ultra-high-resolution measurement of the stability of Fc, AS1 and AS2 assessed by differential scanning fluorimetry (DSF). C, Determination by ELISA of the EC₅₀, of AS1 and AS2 for the recombinant catalytic domain of LOX. D, Determination by ELISA of the EC₅₀ of AS1 and AS2 for recombinant HSP70. E, Supernatants from mouse melanoma cell line Ret after two days of culture were collected, concentrated 10-fold, immunoprecipitated using AS1 and AS2 (Fc was used as a control), and the immunoprecipitated proteins were detected by Western blot using anti-LOX and anti-HSP70 antibodies. F, Volcano plot of proteomics analysis of HDF proteins co-immunoprecipitated with AS1 inhibitor, Fc immunoprecipitated samples were used as a control (AS1/Fc). G, Volcano plot of proteomics analysis of HDF supernatants proteins that co-immunoprecipitated with AS2 inhibitor, Fc immunoprecipitated samples were used as a control (AS2/Fc). H, Proteins immunoprecipitated with AS1 and AS2 from the culture media of human dermal fibroblasts (Fc was used as a control), were analyzed by Western blot using anti-LOX antibody. I, Representative two photon-SHG images of collagen fibers deposited by HDFs after a 7-day treatment with Fc, AS1 and AS2. J, Quantification of orientation entropy of collagen fibers after treatment with Fc, AS1 (NS) and AS2 (**p<0.0020). K, Quantification of collagen thickness after a 7-day culture of HDFS in presence of additives stimulating ECM production and 5 μM Fc, AS1 (*p<0.0417) or AS2 (**p<0.0030). L, ATPase activity of HSP70 measured in the media of Ret melanoma cells after treatment with 5 PM Fc, AS1 or AS2. M, ATPase activity of HSP70 measured in lysates of Ret melanoma cells after treatment with 5 μM Fc, AS1 or AS2. Data are presented as mean±SEM; values of p<0.05 were considered statistically significant (*p<0.05, **p<0.01, ***p<0.001, NS: not significant).

FIGS. 3A-I: Effect of AS1 and AS2 on the anti-metastatic potential on human and mice melanoma cell lines and when combined with immune checkpoint inhibitors.

Effect of AS1 and AS2 on in vitro invasion assay performed with A. human melanoma cell lines (WM3314 and WM1716). B, mouse B16-F10 melanoma cell line. C, mouse Ret melanoma cell line. D, Representative images of B16-F10 and Ret melanoma cells captured during the invasion assays. E, Migration assay (wound healing) of B16-F10 melanoma cells during a 24-h treatment with AS1, AS2 and Fc as a control. F-I, Immunofluorescence staining of AS1- and AS2-treated Ret melanoma cells with cytoskeleton and mesenchymal markers, as well as their quantification graphs. Scale bars: 20 nm

FIGS. 4A-H: In vivo evaluation of AS1 and AS2 effect in melanoma to lung metastasis mouse model.

A, The melanoma to lung metastasis mouse model. In this experimental process 0.5×10⁶ Ret melanoma cells were injected in the tail vein. After 5 days 5 mg/kg AS1 and AS2 were injected I.P, this treatment was repeated every other day for 3 weeks. In vivo bioluminescence imaging was performed weekly in order to monitor the tumor progression. After sacrificing the mice at the end of the experiment, tissues were dissected and ex vivo bioluminescence imaging was performed. B, In vivo luciferase imaging of mice was performed weekly to monitor melanoma metastasis formation while mice were treated with 5 mg/kg Fc, AS1 and AS2. C, Quantification of in vivo luciferase imaging during the 3 weeks of the experiment. D, Ex vivo luciferase imaging of lung tissues at the end of the experiment. E, Quantification of average radiance in ex vivo luciferase imaging of lung tissues. F, Quantification after FACS analysis of circulating melanoma cells of mice after 3 weeks of treatment. G, FACS analysis of circulating melanoma cells in mice after 3 weeks of Fc, AS1 and AS2 treatment. H, H&E staining of lungs tissues after 3 weeks of treatment with Fc, AS1 and AS2. In dark purple area the formation of a tumor can be observed after melanoma cell metastasis to lungs. At higher magnification the areas where melanin is secreted around melanoma cells was visible, mainly in Fc control. Data are presented as mean±SEM; values of p<0.05 were considered statistically significant (+P<0.05, **P<0.01, ***P<0.001).

FIGS. 5A-R: Investigation of the role of AS1 and AS2 in the regulation of mice immune system.

A, Protein levels of LOX and HSP70 in the different lung cell types of Fc-treated mouse metastatic lung were analysed by CyTOF. B, Percentage of cells co-expressing LOX and HSP70 in each immune cell population, including melanoma cells. c, CyTOF analysis of the entire melanoma metastatic lung tissue after 3 weeks of treatment with AS1 and AS2, in which the overview of all the cell population present was determined. NK-Natural killer cells, CTLs-cytotoxic T cells (CD8+ T cells), THL-T-helper cells (CD4+ T cells), AEC-Alveolar epithelial cells, LEPs-Late endothelial progenitors, EEPs-Early endothelial progenitors. Number of mice used in each group, Fc (n=7), AS1 (n=6) and AS2 (n=7). D, Proliferation rate of melanoma cells isolated from lung after staining with Ki-67 and CyTOF analysis. E, Quantification of Ki-67 proliferation marker in metastatic melanoma cells. F, The percentage of live melanoma cells after treatment with AS1 and AS2 as well as the percentage of LOX+/HSP70+ melanoma cells before and after the treatment with AS1 and AS2. G, The percentage of total immune cells after treatment with AS1 and AS2 and their effect on LOX+/HSP70+ total immune cells. H, The percentage of CD4+ T-cells after treatment with AS1 and AS2 and their effect on LOX+/HSP70+CD4+ T-cells. i, The percentage of T-cells after treatment with AS1 and AS2 and their effect on LOX+/HSP70+ T-cells. J, Representative images of CyTOF after gating of double positive CD8⁺ T-cells treated with AS1 and AS2. K, Representative images of CyTOF after gating of double positive CD4⁺CD8⁺ T-cells treated with AS1 and AS2. L, Quantification of CD4⁺CD8⁺ T-cells treated with AS1 and AS2. M, The percentage of dendritic cells treated with AS1 and AS2 and their effect on LOX+/HSP70+ dendritic cells. N, Percentage of LOX+/HSP70+ dendritic cells compared to total cells. Orange lines represent the median values; outer lines represent the interquartile range. O, The percentage of macrophages treated with AS1 and AS2 and their effect on LOX+/HSP70+ macrophages. P, Percentage of LOX+/HSP70+ macrophages compared to total cells. Orange lines represent the median values; outer lines represent the interquartile range. Q, Killing assay of Ret-melanoma cells co-cultured with isolated CD8⁺ T cells from tumor inoculated spleen in the presence of Fc, AS1 and AS2 alone or in combination with anti-PD1 antibody. R, Representative images of viable melanoma cells after 4 days of co-treatment with anti-PD1 antibody and AS1 or AS2. Data are presented as mean±SEM; values of p<0.05 were considered statistically significant (*P<0.05, **P<0.01, ***P<0.001).

FIGS. 6A-F: Effect of AS1 and AS2 on genomic analysis of metastatic melanoma cells. A, Representative images of lung tissue (upper panel side) and melanoma tumor (lower panel side) after their separation. B, Heatmap of RNA-seq results of all genes on the subset of genes related to migration and differentiation. Upregulated genes are in red and downregulated genes in blue. C, Functional enrichment analysis performed with STRING to compare AS1-treated melanoma cells to Fc-treated melanoma cells, with average log fold change values associated to each gene. Downregulated pathway of AS1-treated melanoma cells by Kyoto Encyclopedia of Genes and Genomes (KEGG) database. D, Functional enrichment analysis performed with STRING to compare AS2-treated melanoma cells to Fc-treated melanoma cells and downregulated pathways of AS2-treated melanoma cells by KEGG. E, Functional enrichment analysis of biological pathways performed by STRING to compare AS1-treated melanoma cells to Fc-treated melanoma cells and downregulated pathways of AS1-treated melanoma cells. F, Functional enrichment analysis of biological pathways performed with STRING to compare AS2-treated melanoma cells to Fc-treated melanoma cells and downregulated pathways of AS2-treated melanoma cells. The pathways were curated by Reactome.

FIGS. 7A-E: AS1 and AS2 decrease the expression of ECM degradation enzymes and of an EMT biomarker.

A, Percentage of the expression levels of LOX, HSP70 and MMP2 in lung metastatic melanoma cells measured by CyTOF analysis, before and after treatment with AS1, AS2 and Fc. B, Percentage of the expression levels of LOX and HSP70, as well as MMP2, MMP7 and ADAM17 in macrophages of metastatic lung tissue measured by CyTOF. C, Percentage of the expression levels of ADAM17, MMP7 and HSP70 in lung metastatic T-cells measured by CyTOF, before and after the treatment with AS1, AS2 and Fc. D, Percentage of the expression levels of LOX, HSP70, ADAM17, MMP2 and MMP7 in dendritic cells of metastatic lung tissue measured by CyTOF. E, Western blot image (upper panel) and quantitative analysis (lower panel) showing the expression of FAM3C/ILEI by lung metastatic melanoma cells from after treatment with AS1, AS2 and Fc. Data are presented as mean±SEM; values of p<0.05 were considered statistically significant (*P<0.05, **P<0.01, ***P<0.001).

FIGS. 8A-D: Identification of novel tumor targets of AS1 and AS2 using a tissue array. A, Quantification of fluorescence intensity of AS1 binding on 24 human cancer types. Red lines represent the median values; black lines represent the interquartile range. B, Quantification of fluorescence intensity of AS2 binding on 24 human cancer types. Red lines represent the median values; black lines represent the interquartile range. C, Representative images of four tumors with gradient affinity to AS1 and AS2, which is aligned to expression levels of LOX and HSP70. D, Schematic representation of tumor microenvironment rearrangement after using AS1 and AS2 decoys in metastatic mouse melanoma model. Scale bars, 100 μm.

FIGS. 9A-C: Expression levels of ECM remodeling enzymes and protein sequence of AS1 inhibitor.

A, Schematic representation of amino acid sequence of AS1 inhibitor (SEQ ID NO: 12). Marked are the N-glycosylation sites of LOX-PP sequence which are mutated on AS2 inhibitor. B, Expression levels of matrix remodeling enzymes by metastatic melanoma cells. C, Quantification of MS analysis after immunoprecipitation of HDF cells supernatants with Fc, AS1 and AS2-coated beads.

FIGS. 10A-B: H&E and immunofluorescence staining on lung tissues of AS1 and AS2 treated mice.

A, Distribution of LOX and HSP70 biomarkers at the lung tissues is presented after immunofluorescence staining with anti-LOX and anti-HSP70 antibodies. Localization of LOX and HSP70 was present in melanoma and non-melanoma tissue areas. B, Evaluation of AS1 and AS2 ability to bind in melanoma cells in situ was performed in order to confirm that LOX and HSP70 were recognized in vivo by AS1 and AS2. In the upper panel tissues biotinylated AS1 was used for staining and streptavidin-conjugated to Cyanine Cy™3 for detection in the lower panel biotinylated AS2 was used for the staining. For both AS1 and AS2 the staining was stronger in the tumor area and co-localized with the LOX protein.

FIGS. 11A-B: Pathway enrichment analysis of metastatic melanoma cells of decoy's treated mice.

(A) RNA-seq results of metastatic melanoma cells of AS1-treated mice used for functional enrichment analysis and the top 20 enriched terms across the differentially expressed genes compared to Fc-treated mice were identified using Metascape. The threshold was considered Log|FC|>0.6 and presented in the graph colored by p values.
(B) Pathway enrichment analysis of metastatic melanoma cells after treatment with AS2 inhibitor compare to Fc. Top 20 enriched terms across the differentially expressed genes were produced using Metascape. As a cut-off criterion was considered the following: Log|FC|>0.6 and presented in the graph colored by p values.

### DESCRIPTION OF SPECIFIC EMBODIMENTS OF THE INVENTION

The present invention, in some embodiments thereof, relates to methods of treating cancer which are characterized by an up-regulation in the extracellular matrix molecules lysyl oxidase (LOX) and heat-shock protein 70 (HSP70).

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Existing immune and targeted therapies on advanced melanoma have approximately 50% response on efficacy due to resistance to treatment. However, cancer is a fibrotic disease and the excessive extracellular matrix (ECM) turnover drives disease progression, immune suppression and affects treatment response.

The present inventors propose co-targeting two melanoma markers, lysyl oxidase and heat shock protein 70 (HSP70), whose expression levels are elevated during melanoma progression in human biopsies samples. The present inventors developed two lysyl oxidase prodomain inhibitors, each fused to Fc antibody fragment (referred to herein as AS1 & AS2) which are capable of binding both lysyl oxidase and HSP70.

Thorough characterization of inhibitors proved their high binding affinity and inhibitory capacity against both biomarkers (FIGS. 2A-M). In addition, bispecific inhibitors diminished the circulated melanoma cells and led to activation of both innate and adaptive immune system (FIGS. 3A-F). Combinational treatment with immune checkpoint inhibitor dramatically increased the CD8+ T-cell-induced cytotoxicity (FIGS. 3G-I). These findings suggest that targeting LOX and HSP70 biomarkers has the potential to be a new strategy for melanoma treatment and for enhancing the efficacy of existing immunotherapies. Such treatments can be adapted for other cancers which are characterized by an increase in LOX and HSP70 expression.

Thus, according to an aspect of the present invention, there is provided a method of treating a cancer selected from the group consisting of melanoma, prostate adenocarcinoma, testis embryonal carcinoma, ovarian cancer, uterus carcinoma, pancreatic adenocarcinoma, astrocytoma and glioblastoma comprising administering to the subject a therapeutically effective amount of a polypeptide comprising a propeptide of lysyl oxidase (LOX), the polypeptide being devoid of LOX catalytic activity, wherein the polypeptide binds to both LOX and heat shock protein 70 (HSP70) with a EC₅₀ of less than 100 nM, thereby treating the cancer.

According to another aspect of the present invention, there is provided a method of treating a cancer which is characterized by an up-regulation of expression of lysyl oxidase (LOX) and heat shock protein 70 (HSP70), the method comprising administering to the subject a therapeutically effective amount of a polypeptide comprising a propeptide of lysyl oxidase (LOX), the polypeptide being devoid of LOX catalytic activity, wherein the polypeptide binds to both LOX and heat shock protein 70 (HSP70) with a EC₅₀ of less than 100 nM, thereby treating the cancer.

As used herein “treating” refers to abrogating, substantially inhibiting, slowing or reversing the progression of a condition, substantially ameliorating clinical or aesthetical symptoms of a condition or substantially preventing the appearance of clinical or aesthetical symptoms of a condition.

As used herein the term “subject” refers to a subject diagnosed with a cancer or predicted to have the cancer. In one embodiment, the subject is selected for the treatment based on the level of LOX and HSP70 in a sample derived from the subject. When the level of LOX and HSP70 is above a predetermined amount (for example at least 1.5 times, 2 times, 5 times or more) than the amount in a sample of the same type derived from a subject known not to have the cancer (e.g. a healthy subject), the subject is deemed as being a suitable candidate for the treatment. Methods of measuring LOX and HSP70 are described herein below.

The polypeptides disclosed herein are not occurring in nature, (i.e. synthetic) either because they are isolated from a natural environment thereof e.g., the human or animal body, or because they are mutated with respect to the wild-type form or because they are modified e.g., attached to a heterologous moiety e.g., protein or chemical.

As used herein “Lysyl Oxidase” or “LOX” refers to the protein product of the LOX gene. In humans, the LOX gene is located on chromosome 5923.3-31.2. The primary sequence of the LOX protein is highly conserved in mammals. Human LOX is synthesized as a pre-proprotein (pre-pro-LOX) of 417 amino acids (UniProtKB-P28300, which undergoes a number of post-translational modifications within the endoplasmic reticulum (ER) and post-ER e.g., glycosylation as described below. After cleavage of the 21 amino acid signal sequence, the N-terminal propeptide, comprising 147 amino acid residues, is N-glycosylated and the C-terminal sequence containing the 249 amino acid residue mature protein which is also referred to herein as the part which comprises the LOX catalytic activity, is distinctively folded to acquire at least three disulfide bonds. Copper is a cofactor of the functional catalyst, incorporated into the nascent enzyme within the ER. The enzyme also contains a peptidyl organic cofactor, lysyltyrosine quinone (LTQ) generated by an intramolecular cross-link between lysine 320 and the copper-dependent oxidation product of tyrosine 355.

The “LOX-propeptide” (LOX-PP) or “LOX-prodomain” (LPD) is the N-terminal propeptide corresponding to amino acid residues 22-168 of SEQ ID NO: 9, which following secretion of proLOX to the extracellular space, is cleaved by procollagen-C-proteinase (BMP-1) or BMP-1-related metalloproteinases, to generate the free propeptide (LPD) and the catalytically active LOX.

A key intracellular function of LOX-PP (LPD) is most likely the maintenance of lysyl oxidase in an inactive state within the secretory pathway. Propeptides may also function as intramolecular chaperones to facilitate correct folding and the eventual targeting of these proteins to their destinations.

The LOX-PP comprises three consensus N-glycosylation sites at residues 81, 97, and 144 of the LOX protein sequence.

The present teachings contemplate, in one embodiment, a glycosylated LPD.

According to an embodiment, the polypeptide is glycosylated on at least one of N81, N97 and N144 of SEQ ID NO: 1. When referring to the glycosylation sites the indicated residues are those corresponding to SEQ ID NO: 1.

According to an embodiment, the polypeptide is glycosylated on one glycosylation site of LPD i.e., N81 of SEQ ID NO: 1.

According to an embodiment, the polypeptide is glycosylated on one glycosylation site of LPD i.e., N97 of SEQ ID NO: 1.

According to an embodiment, the polypeptide is glycosylated on one glycosylation site of LPD i.e., N144 of SEQ ID NO: 1.

According to an embodiment, the polypeptide is glycosylated on at least two of N81, N97 and N144 of SEQ ID NO: 1.

According to an embodiment, the polypeptide is glycosylated on two glycosylation sites of LPD, e.g., N81+N97 of SEQ ID NO: 1, e.g., N81+N144 of SEQ ID NO: 1 e.g., N97+N144 of SEQ ID NO: 1.

According to an embodiment, the polypeptide is glycosylated on N81, N97 and N144 of SEQ ID NO: 1.

According to an alternative embodiment, the polypeptide is not glycosylated on N81, N97 and N144 of SEQ ID NO: 1.

According to an alternative embodiment, the polypeptide comprises a sequence as set forth in SEQ ID NO: 4.

Alteration in glycosylation can be achieved by methods which are well known in the art e.g., any of selection of an appropriate expression system (e.g., prokaryotic vs. eukaryotic), separation of glycosylated from non-glycosylated forms, site-directed mutagenesis at the glycosylation site and/or using enzymatic modification.

The polypeptide of some embodiments of the present invention is endowed with a number of biological activities.

According to a specific embodiment, the protein inhibits LOX in vitro and in vivo. Fc-LPD affinity and stability measurements were performed against LOX, determining the binding affinity (EC₅₀=40 nM or 43 nM) and dissociation constant (Kd=32 nM).

According to a specific embodiment, the polypeptide is characterized by an EC₅₀ of about 10-2000 nM, about 10-500 nM, e.g., between 40-50 nM, as determined by an ELISA assay.

According to a specific embodiment, the polypeptide is characterized by a K_{D} of about 10-100 nM, e.g., about 32 nM, as determined by a microscale thermophoresis.

According to a particular embodiment, the polypeptide is capable of downregulating crosslinking of collagen in an in vitro assay system. Since the polypeptide of some embodiments of the invention is capable of interfering with collagen crosslinking, it alters the structure of the extracellular matrix (ECM), relaxing the collagen fibers, changing them from an ordered orientation to a more random orientation. Thus, the polypeptide of some embodiments of the present invention alters the fibrillation of collagen (and accordingly strength of the collagen) and affecting the thickness of collagen.

The mRNA and amino acid sequences for Homo sapiens LOX can be found under GenBank accession number P28300.2 (Protein accession number), AF039291.1 (mRNA accession number), NC_000005 (genomic accession).

As mentioned, the polypeptide is devoid of LOX catalytic activity.

As used herein “LOX catalytic activity” refers to the lysyl oxidase activity of the enzyme which is typically attributed to the domain between amino acid coordinates 169-417 of SEQ ID NO: 9 (e.g., SEQ ID NO: 10). According to a specific embodiment the catalytic activity is set forth in SEQ ID NO: 10 (amino acid sequence) and 11 (nucleic acid sequence).

Catalytic activity of LOX can be determined in vitro by SHG microscopy (described in details in the Examples section which follows).

Functional equivalents of LPD are also contemplated, having about the same or even higher activity/stability than that of SEQ ID NOs: 1 or 4.

According to a particular embodiment, the LPD sequence is at least 80%, at least 85%, at least 87%, at least 89%, at least 91%, at least 93%, at least 95% or more say 100% identical to the LPD sequence described in SEQ ID NO: 1 or 5 as determined using BlastP software of the National Center of Biotechnology Information (NCBI) using default parameters) and is capable of any of the above biological functions. In measuring homology between a peptide and a protein of greater size, homology is measured only in the corresponding region; that is, the protein is regarded as only having the same general length as the peptide, allowing for gaps and insertions.

The homolog may also refer to a deletion, insertion, or substitution variant, including an amino acid substitution, thereof and biologically active polypeptide fragments thereof.

Additional modifications and changes can be made in the structure of the LPD portion of the polypeptide of the presently disclosed subject matter and still obtain a molecule being capable of inhibiting LOX. For example, certain amino acids can be substituted for other amino acids in a sequence without appreciable loss of peptide activity. Because it is the interactive capacity and nature of a polypeptide that defines that polypeptide's biological functional activity, certain amino acid sequence substitutions can be made in a polypeptide sequence (or, of course, its underlying DNA coding sequence) and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and the like. It is known in the art that an amino acid can be substituted by another amino acid having a similar hydropathic index and still obtain a functionally equivalent polypeptide. In such changes, the substitution of amino acids whose hydropathic indices are within ±2 is preferred, those which are within ±1 are particularly preferred, and those within ±0.5 are even more particularly preferred.

Substitution of like amino acids can also be made on the basis of hydrophilicity, particularly where the biological functional equivalent polypeptide or peptide thereby created is intended for use in immunological embodiments. U.S. Pat. No. 4,554,101, incorporated herein by reference, states that the greatest local average hydrophilicity of a polypeptide, as governed by the hydrophilicity of its adjacent amino acids, correlates with its immunogenicity and antigenicity, i.e. with a biological property of the polypeptide. As detailed in U.S. Pat. No. 4,554,101, the following hydrophilicity values have been assigned to amino acid residues: arginine (+3.0); lysine (+3.0); aspartate (+3.0.+−. 1); glutamate (+3.0.+−. 1); serine (+0.3); asparagine (+0.2); glutamine (+0.2); glycine (0); proline (−0.5.+−. 1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those which are within ±1 are particularly preferred, and those within ±0.5 are even more particularly preferred.

As outlined above, amino acid substitutions are generally therefore based on the relative similarity of the amino acid side-chain substituents, for example, their hydrophobicity, hydrophilicity, charge, size, and the like. Exemplary substitutions which take various of the foregoing characteristics into consideration are well known to those of skill in the art and include: arginine and lysine; glutamate and aspartate; serine and threonine; glutamine and asparagine; and valine, leucine and isoleucine. The presently disclosed subject matter thus contemplates functional or biological equivalents of the polypeptide or LPD portion thereof as set forth above.

Biological or functional equivalents of a polypeptide can be prepared using site-specific mutagenesis according to procedures well known in the art. Accordingly, amino acid residues can be added to or deleted from the LPD of the presently disclosed subject matter through the use of standard molecular biological techniques without altering the functionality of the peptide.

According to one embodiment, the amino acid sequence of the LPD is modified so as to increase its stability, bioavailability and/or pharmacological efficacy.

Thus, according to an aspect of the invention there is provided a synthetic polypeptide comprising a propeptide of human lysyl oxidase (LOX), the polypeptide being devoid of LOX catalytic activity, the synthetic polypeptide comprising a modification which imparts the polypeptide with enhanced stability under physiological conditions as compared to a native form of the polypeptide not comprising the modification.

Methods of determining stability are well known in the art.

As used herein “stability” refers to at least thermal stability. The method is based on measuring ultra-high-resolution protein stability using intrinsic tryptophan or tyrosine fluorescence.

As used herein “enhanced” or “increased” refers to an increase by at least 10%, 20%, 30%, 50%, 60%, 70%, 80%, 90% or more, say 100%, with respect to that of the native LPD.

According to a specific embodiment, the polypeptide is characterized by a transition midpoint of 20-95° C. e.g., Tm about 70° C., as determined by differential scanning fluorimetry (DSF).

Thus, in order to improve the stability/activity of the LPD, the polypeptide is modified.

According to a specific embodiment, the modification comprises a proteinaceous modification.

The proteinaceous modification can be attached to the polypeptide by ways of chemical attachment (fusion polypeptide such as by the use of linkers and/or active groups) or by recombinant DNA technology, whereby the synthetic polypeptide is a chimeric polypeptide.

Thus, the polypeptide has a first moiety, which is the LPD, and a second moiety, which is a heterologous peptide or protein, i.e., the proteinaceous modification. The fusion/chimera (or collectively “fusion”) can be with N to C or C to N orientation of the LPD relative to the proteinaceous moiety (also referred to herein as “heterologous polypeptide”). Fusion proteins may include myc, HA-, or His6-tags. Fusion proteins further include the LPD fused to the Fc domain of a human IgG (as referred to herein in one embodiment Fc-LPD). In particular aspects, the immunoglobulin fusion includes the hinge, CH2 and CH3, or the hinge, CH1, CH2 and CH3 regions of an IgG1 molecule. According to a specific embodiment, the Fc is as set forth in SEQ ID NO: 7. For the production of immunoglobulin fusions see also U.S. Pat. No. 5,428,130. The Fc moiety can be derived from mouse IgG1 or human IgG24. Human IgG2M4 (See U.S. Published application No. 20070148167 and U.S. Published application No. 20060228349) is an antibody from IgG2 with mutations with which the antibody maintains normal pharmacokinetic profile but does not possess any known effector function.

Exemplary amino acid sequences of an LPD fused to an Fc domain is set forth in SEQ ID NO: 2 or 5 (amino acid, with the signal sequence) and SEQ ID NO: 12 or 13 (without the signal sequence). Exemplary nucleic acid sequences encoding same are set forth in SEQ ID NOs: 3 or 6.

Fusion proteins further include the LPD fused to human serum albumin, transferrin, or an antibody.

In further still aspects, the LPD is conjugated to a carrier protein such as human serum albumin, transferrin, or an antibody molecule.

The term “polypeptide” as used herein refers to a polymer of natural or synthetic amino acids, encompassing native peptides (either truncation products, synthetically synthesized polypeptides or recombinant polypeptides) and peptidomimetics (typically, synthetically synthesized peptides), as well as peptoids and semipeptoids which are polypeptide analogs, which may have, for example, modifications rendering the polypeptides even more stable while in a body or more capable of penetrating into cells.

Such modifications include, but are not limited to N terminus modification, C terminus modification, polypeptide bond modification, including, but not limited to, CH2-NH, CH2-S, CH2-S—O, O—C—NH, CH2-O, CH2-CH2, S═C—NH, CH—CH or CF=CH, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further details in this respect are provided hereinunder.

Polypeptide bonds (—CO—NH—) within the polypeptide may be substituted, for example, by N-methylated bonds (—N(CH3)-CO—), ester bonds (—C(R)H—C—O—O—C(R)—N—), ketomethylen bonds (—CO—CH2-), α-aza bonds (—NH—N(R)—CO—), wherein R is any alkyl, e.g., methyl, carba bonds (—CH2-NH—), hydroxyethylene bonds (—CH(OH)—CH2-), thioamide bonds (—CS—NH—), olefinic double bonds (—CH—CH—), retro amide bonds (—NH—CO—), polypeptide derivatives (—N(R)—CH2-CO—), wherein R is the “normal” side chain, naturally presented on the carbon atom.

These modifications can occur at any of the bonds along the polypeptide chain and even at several (2-3) at the same time.

Natural aromatic amino acids, Trp, Tyr and Phe, may be substituted for synthetic non-natural acid such as Phenylglycine, TIC, naphthylalanine (Nol), ring-methylated derivatives of Phe, halogenated derivatives of Phe or o-methyl-Tyr.

In addition to the above, the polypeptides of the present invention may also include one or more modified amino acids or one or more non-amino acid monomers (e.g. fatty acids, complex carbohydrates etc.).

As used herein in the specification and in the claims section below the term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids (stereoisomers).

Tables A and B below list naturally occurring amino acids (Table A) and non-conventional or modified amino acids (Table B) which can be used with the present invention.

TABLE A

| | Three-Letter | One-letter |
|---|---|---|
| Amino Acid | Abbreviation | Symbol |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic Acid | Glu | E |
| Glycine | Gly | G |
| Histidine | His | H |
| Isoleucine | Iie | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any amino acid as above | Xaa | X |

TABLE B

| Non-conventional | | Non-conventional | |
|---|---|---|---|
| amino acid | Code | amino acid | Code |
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| Carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgin |
| Carboxylate | | L-N-methylglutamic acid | Nmglu |
| Cyclohexylalanine | Chexa | L-N-methylhistidine | Nmhis |
| Cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α ethylcyclohexylalanine | Mchexa |
| D-α-methylarginine | Dmarg | α-methylcyclopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cyclododeclglycine | Ncdod |
| D-α-methylalnine | Dnmala | N-cyclooctylglycine | Ncoct |
| D-α-methylarginine | Dnmarg | N-cyclopropylglycine | Ncpro |
| D-α-methylasparagine | Dnmasn | N-cycloundecylglycine | Ncund |
| D-α-methylasparatate | Dnmasp | N-(2,2-diphenylethyl)glycine | Nbhm |
| D-α-methylcysteine | Dnmcys | N-(3,3-diphenylpropyl)glycine | Nbhe |
| D-N-methylleucine | Dnmleu | N-(3-indolylyethyl) glycine | Nhtrp |
| D-N-methyllysine | Dnmlys | N-methyl-y-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nva |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | Penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-αthylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl)glycine | Nser |
| D-N-methylisoleucine | Dnmile | N-(imidazolylethyl)glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | Penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α ethylhistidine | Mhis | L-α-methylhomophenylalanine | Mhphe |
| L-α thylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | Mser | L-α-methylthreonine | Mthr |
| L-α ethylvaline | Mtrp | L-α-methyltyrosine | Mtyr |
| L-α-methylleucine | Mval | L-N-methylhomophenylalanine | Nmhphe |
| | Nnbhm | | |
| N-(N-(2,2-diphenylethyl) | | N-(N-(3,3-diphenylpropyl) | |
| carbamylmethyl-glycine | Nnbhm | carbamylmethyl(1)glycine | Nnbhe |
| 1-carboxy-1-(2,2-diphenyl | Nmbc | | |
| ethylamino)cyclopropane | | | |

Recombinant techniques are typically used to generate the polypeptides (or only the LPD portion thereof) of the present invention. Such recombinant techniques are described by Bitter et al., (1987) Methods in Enzymol. 153:516-544, Studier et al. (1990) Methods in Enzymol. 185:60-89, Brisson et al. (1984) Nature 310:511-514, Takamatsu et al. (1987) EMBO J. 6:307-311, Coruzzi et al. (1984) EMBO J. 3:1671-1680 and Brogli et al., (1984) Science 224:838-843, Gurley et al. (1986) Mol. Cell. Biol. 6:559-565 and Weissbach & Weissbach, 1988, Methods for Plant Molecular Biology, Academic Press, NY, Section VIII, pp 421-463.

To produce a polypeptide of the present invention using recombinant technology, a polynucleotide encoding the polypeptide of the present invention e.g., SEQ ID NO: 3 or 6 is ligated into a nucleic acid expression vector, which comprises the polynucleotide sequence under the transcriptional control of a cis-regulatory sequence (e.g., promoter sequence) suitable for directing constitutive, tissue specific or inducible transcription of the polypeptides of the present invention in the host cells.

The phrase “an isolated polynucleotide” refers to a single or double stranded nucleic acid sequence which is isolated and provided in the form of an RNA sequence, a complementary polynucleotide sequence (cDNA), a genomic polynucleotide sequence and/or a composite polynucleotide sequences (e.g., a combination of the above).

As used herein the phrase “complementary polynucleotide sequence” refers to a sequence, which results from reverse transcription of messenger RNA using a reverse transcriptase or any other RNA dependent DNA polymerase. Such a sequence can be subsequently amplified in vivo or in vitro using a DNA dependent DNA polymerase.

As used herein the phrase “genomic polynucleotide sequence” refers to a sequence derived (isolated) from a chromosome and thus it represents a contiguous portion of a chromosome.

As used herein the phrase “composite polynucleotide sequence” refers to a sequence, which is at least partially complementary and at least partially genomic. A composite sequence can include some exonal sequences required to encode the polypeptide of the present invention, as well as some intronic sequences interposing therebetween. The intronic sequences can be of any source, including of other genes, and typically will include conserved splicing signal sequences. Such intronic sequences may further include cis acting expression regulatory elements.

As mentioned hereinabove, polynucleotide sequences of the present invention are inserted into expression vectors (i.e., a nucleic acid construct) to enable expression of the recombinant polypeptide. The expression vector of the present invention may include additional sequences which render this vector suitable for replication and integration in prokaryotes, eukaryotes, or preferably both (e.g., shuttle vectors). Typical cloning vectors contain transcription and translation initiation sequences (e.g., promoters, enhances) and transcription and translation terminators (e.g., polyadenylation signals). It will be appreciated that the expression vector may also comprise polynucleotide sequences encoding other polypeptides that are transcriptionally linked to the nuclear targeting peptides of the present invention. Such polypeptides are further described herein below.

Promoters used in the expression vectors may be constitutive or inducible. Tissue specific promoters are also contemplated.

A variety of prokaryotic or eukaryotic cells (e.g., HEK293-6E) can be used as host-expression systems to express the peptides of the present invention. These include, but are not limited to, microorganisms, such as bacteria transformed with a recombinant bacteriophage DNA, plasmid DNA or cosmid DNA expression vector containing the polypeptide coding sequence; yeast transformed with recombinant yeast expression vectors containing the polypeptide coding sequence; plant cell systems infected with recombinant virus expression vectors (e.g., cauliflower mosaic virus, CaMV; tobacco mosaic virus, TMV) or transformed with recombinant plasmid expression vectors, such as Ti plasmid, containing the polypeptide coding sequence.

According to this embodiment of this aspect of the present invention, the nucleic acid sequence encoding the polypeptide of the present invention may be altered, to further improve expression levels in the expression system. Thus, the polynucleotide sequence encoding the polypeptide may be modified in accordance with the preferred codon usage for bacterial or a certain mammalian expression.

The phrase “codon optimization” refers to the selection of appropriate DNA nucleotides for use within a structural gene or fragment thereof that approaches codon usage within the relevant system.

Examples of polynucleotide sequences that may be used to express the polypeptides of the present invention are provided in SEQ ID NOs: 3 and 6.

It will be appreciated that the polynucleotides of the present invention may also be expressed directly in the subject (i.e. in vivo gene therapy) or may be expressed ex vivo in a cell system (autologous or non-autologous) and then administered to the subject.

Other than containing the necessary elements for the transcription and translation of the inserted coding sequence (encoding the polypeptide), the expression construct of the present invention can also include sequences engineered to optimize stability, production, purification, yield or activity of the expressed peptide.

Various methods can be used to introduce the expression vector of the present invention into the host cell system. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

Transformed cells are cultured under effective conditions, which allow for the expression of high amounts of recombinant polypeptide. Effective culture conditions include, but are not limited to, effective media, bioreactor, temperature, pH and oxygen conditions that permit protein production. An effective medium refers to any medium in which a cell is cultured to produce the recombinant polypeptide of the present invention. Such a medium typically includes an aqueous solution having assimilable carbon, nitrogen and phosphate sources, and appropriate salts, minerals, metals and other nutrients, such as vitamins. Cells of the present invention can be cultured in conventional fermentation bioreactors, shake flasks, test tubes, microtiter dishes and petri plates. Culturing can be carried out at a temperature, pH and oxygen content appropriate for a recombinant cell. Such culturing conditions are within the expertise of one of ordinary skill in the art.

Depending on the vector and host system used for production, resultant polypeptides of the present invention may either remain within the recombinant cell, secreted into the fermentation medium, secreted into a space between two cellular membranes, such as the periplasmic space in E. coli; or retained on the outer surface of a cell or viral membrane.

Following a predetermined time in culture, recovery of the recombinant polypeptide is affected.

The phrase “recovering the recombinant polypeptide” used herein refers to collecting the whole fermentation medium containing the polypeptide and need not imply additional steps of separation or purification.

Recovering is also covered by the term “isolating” or “purifying”, which can also be from the host cells.

Thus, polypeptides of the present invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization.

To facilitate recovery, the expressed coding sequence can be engineered to encode the polypeptide of the present invention and fused cleavable moiety. Such a fusion protein can be designed so that the polypeptide can be readily isolated by affinity chromatography; e.g., by immobilization on a column specific for the cleavable moiety. Where a cleavage site is engineered between the polypeptide and the cleavable moiety, the polypeptide can be released from the chromatographic column by treatment with an appropriate enzyme or agent that specifically cleaves the fusion protein at this site [e.g., see Booth et al., Immunol. Lett. 19:65-70 (1988); and Gardella et al., J. Biol. Chem. 265:15854-15859 (1990)].

Exemplary purification tags for purposes of the invention include but are not limited to polyhistidine, V5, myc, protein A, gluthatione-S-fransferase, maltose binding protein (MBP) and cellulose-binding domain (CBD) [Sassenfeld, 1990, TIBTECH, 8, 88-9].

The polypeptides of the present invention are preferably retrieved in “substantially pure” form.

As used herein, the phrase “substantially pure” refers to a purity that allows for the effective use of the protein in the applications described herein.

The LPD or polypeptide (including e.g., the Fc portion) of some embodiments of the invention may be chemically modified with a chemical modification following expression for increasing bioavailability.

Thus, for example, the present invention contemplates modifications wherein the polypeptide is linked to a polymer. The polymer selected is usually modified to have a single reactive group, such as an active ester for acylation or an aldehyde for alkylation, so that the degree of modification may be controlled. Included within the scope of polymers is a mixture of polymers. Preferably, for therapeutic use of the end-product preparation, the polymer will be pharmaceutically acceptable.

The polymer or mixture thereof may be selected from the group consisting of, for example, polyethylene glycol (PEG), monomethoxy-polyethylene glycol, dextran, cellulose, or other carbohydrate based polymers, poly-(N-vinyl pyrrolidone) polyethylene glycol, propylene glycol homopolymers, a polypropylene oxide/ethylene oxide co-polymer, polyoxyethylated polyols (for example, glycerol), and polyvinyl alcohol.

In further still embodiments, the polypeptide is modified by PEGylation, HESylation CTP (C terminal peptide), crosslinking to albumin, encapsulation, modification with polysaccharide and polysaccharide alteration. The modification can be to any amino acid residue in the polypeptide.

According to one embodiment the modification is to the N or C-terminal amino acid of the LPD. This may be affected either directly or by way coupling to the thiol group of a cysteine residue added to the N or C-terminus or a linker added to the N or C-terminus such as Ttds. In further embodiments, the N or C-terminus of the polypeptide comprises a cysteine residue to which a protecting group is coupled to the N-terminal amino group of the cysteine residue and the cysteine thiolate group is derivatized with a functional group such as N-ethylmaleimide, PEG group, HESylated CTP.

It is well known that the properties of certain proteins can be modulated by attachment of polyethylene glycol (PEG) polymers, which increases the hydrodynamic volume of the protein and thereby slows its clearance by kidney filtration. (See, for example, Clark et al., J. Biol. Chem. 271:21969-21977 (1996). Therefore, it is envisioned that the core peptide residues can be PEGylated to provide enhanced therapeutic benefits such as, for example, increased efficacy by extending half-life in vivo. Thus, PEGylating the polypeptide will improve the pharmacokinetics and pharmacodynamics of the propeptide domain of the polypeptide.

PEGylation methods are well known in the literature and described in the following references, each of which is incorporated herein by reference: Lu et al., Int. J. Pept. Protein Res. 43:127-38 (1994); Lu et al., Pept. Res. 6:140-6 (1993); Felix et al., Int. J. Pept. Protein Res. 46:253-64 (1995); Gaertner et al., Bioconjug. Chem. 7:38-44 (1996); Tsutsumi et al., Thromb. Haemost. 77:168-73 (1997); Francis et al., Int. J. Hematol. 68:1-18 (1998); Roberts et al., J. Pharm. Sci. 87:1440-45 (1998); and Tan et al., Protein Expr. Purif. 12:45-52 (1998). Polyethylene glycol or PEG is meant to encompass any of the forms of PEG that have been used to derivatize other proteins, including, but not limited to, mono-(C.sub.1-10) alkoxy or aryloxy-polyethylene glycol. Suitable PEG moieties include, for example, 40 kDa methoxy poly(ethylene glycol) propionaldehyde (Dow, Midland, Mich.); 60 kDa methoxy poly(ethylene glycol) propionaldehyde (Dow, Midland, Mich.); 40 kDa methoxy poly(ethylene glycol) maleimido-propionamide (Dow, Midland, Mich.); 31 kDa alpha-methyl-w-(3-oxopropoxy), polyoxyethylene (NOF Corporation, Tokyo); mPEG.sub.2-NHS-40 k (Nektar); mPEG2-MAL-40 k (Nektar), SUNBRIGHT GL2-400MA ((PEG).sub.240 kDa) (NOF Corporation, Tokyo), SUNBRIGHT ME-200MA (PEG20 kDa) (NOF Corporation, Tokyo). The PEG groups are generally attached to the LPD polypeptide via acylation, amidation, thioetherification or reductive alkylation through a reactive group on the PEG moiety (for example, an aldehyde, amino, carboxyl or thiol group) to a reactive group on the polypeptide (for example, an aldehyde, amino, carboxyl or thiol group).

The PEG molecule(s) may be covalently attached to any Lys or Cys residue at any position in the polypeptide. Other amino acids that can be used are Tyr and His. Optional are also amino acids with a Carboxylic side chain. The polypeptide described herein can be PEGylated directly to any amino acid at the N-terminus by way of the N-terminal amino group. A “linker arm” may be added to the polypeptide to facilitate PEGylation. PEGylation at the thiol side-chain of cysteine has been widely reported (See, e.g., Caliceti & Veronese, Adv. Drug Deliv. Rev. 55:1261-77 (2003)). If there is no cysteine residue in the polypeptide, a cysteine residue can be introduced through substitution or by adding a cysteine to the N-terminal amino acid. Other options include reagents that add thiols to polypeptides, such as Traut's reagents and SATA.

In particular aspects, the PEG molecule is branched while in other aspects, the PEG molecule may be linear. In particular aspects, the PEG molecule is between 1 kDa and 150 kDa in molecular weight. More particularly, the PEG molecule is between 1 kDa and 100 kDa in molecular weight. In further aspects, the PEG molecule is selected from 5, 10, 20, 30, 40, 50 and 60 kDa.

A useful strategy for the PEGylation of a polypeptide consists of combining, through forming a conjugate linkage in solution, a peptide, and a PEG moiety, each bearing a special functionality that is mutually reactive toward the other. The polypeptide can be easily prepared by recombinant means as described above.

According to one embodiment, the PEG is “preactivated” prior to attachment to the polypeptide. For example, carboxyl terminated PEGs may be transformed to NHS esters for activation making them more reactive towards lysines and N-terminals.

According to another embodiment, the polypeptide is “preactivated” with an appropriate functional group at a specific site. Conjugation of the polypeptide with PEG may take place in aqueous phase or organic co-solvents and can be easily monitored by SDS-PAGE, isoelectric focusing (IEF), SEC and mass spectrometry. The PEGylated polypeptide is then purified. Small PEGs may be removed by ultra-filtration. Larger PEGs are typically purified using anion chromatography, cation chromatography or affinity chromatography. Characterization of the PEGylated polypeptide may be carried out by analytical HPLC, amino acid analysis, IEF, analysis of enzymatic activity, electrophoresis, analysis of PEG: protein ratio, laser desorption mass spectrometry and electrospray mass spectrometry.

Removal of excess free PEG may be performed by packing a column (Tricorn Empty High-Performance Columns, GE Healthcare) with POROS 50 HQ support (Applied Biosystems), following which the column is equilibrated with equilibration buffer (25 mM Tris-HCl buffer, pH 8.2). The PEGylated polypeptide is loaded onto the equilibrated column and thereafter the column is washed with 5CV of equilibration buffer. Under these conditions, the polypeptide binds to the column. PEGylated polypeptide is eluted in the next step by the elution buffer and stored at 2-8° C. for short term, or frozen at −20° C. for long term storage.

The resultant polypeptide may be anywhere between 40-100 KDa, dependent on whether a modification has been added (as described above) and on the glycosylation status (as described above).

For instance, in the case of an Fc fusion:

According to a specific embodiment, the polypeptide is 40-70 KDa.

According to a specific embodiment, the polypeptide is 45-65 KDa.

According to a specific embodiment, the polypeptide is 50-70 KDa.

According to a specific embodiment, the polypeptide is 45 KDa.

According to a specific embodiment, the polypeptide is 65 KDa.

According to a specific embodiment, the polypeptide domain of LOX devoid of the LOX catalytic activity is below 400 amino acids e.g., 100-200, 100-150, 100-300 amino acids long.

The synthetic polypeptide can be used in any method of inhibiting catalytic (enzymatic activity) of LOX, by mere contacting the LOX with the synthetic polypeptide comprising the modification. Such methods can be performed in-vitro, in-vivo or ex-vivo.

Polypeptides of some embodiments of the invention can be used in treating cancers characterized by an up-regulation of expression of lysyl oxidase (LOX) and heat shock protein 70 (HSP70).

Examples of such cancers include, but are not limited to melanoma, prostate adenocarcinoma, testis embryonal carcinoma, ovarian cancer, uterus carcinoma, pancreatic adenocarcinoma, astrocytoma and glioblastoma.

According to a specific embodiment, the cancer is melanoma.

In one embodiment, the cancer is metastasized.

Methods of analyzing the expression of LOX and HSP70 in a sample derived from the subject are known in the art and may be carried out on the protein level or the polynucleotide level.

The sample may be a fluid sample (e.g. blood, urine, semen, saliva, breast milk, aminiotic fluid and cerebrospinal fluid) or a solid sample (e.g. a tumor biopsy).

Typically, analyzing the level of proteins involves the use of antibodies that specifically bind to the particular protein among other methods, including, but not limited to, Enzyme linked immunosorbent assay (ELISA), Western blot, Radio-immunoassay (RIA), Fluorescence activated cell sorting (FACS), Immunohistochemical analyses, In situ and/or in-vitro activity assays and Mass spectrometry based techniques. The antibody may be monoclonal, polyclonal, chimeric, or a fragment of the foregoing, and the step of detecting the protein determinant may be carried out with any suitable immunoassay. Antibodies can be conjugated to a solid support suitable for a diagnostic assay (e.g., beads such as protein A or protein G agarose, microspheres, plates, slides or wells formed from materials such as latex or polystyrene) in accordance with known techniques, such as passive binding. Antibodies as described herein may likewise be conjugated to detectable labels or groups such as radiolabels (e.g., ³⁵S, ¹²⁵I, ¹³¹I), enzyme labels (e.g., horseradish peroxidase, alkaline phosphatase), and fluorescent labels (e.g., fluorescein, Alexa, green fluorescent protein, rhodamine) in accordance with known techniques.

Following is a non-limiting list of examples of methods of determining the expression of the disclosed proteins on the polynucleotide level.

It will be appreciated that when the marker is analyzed on the polynucleotide level, an RNA extract is prepared from the sample. To prepare an RNA extract, the cells of the sample are lysed in an appropriate buffer. The buffer typically includes phenol and/or guanidine isothiocyanate. The buffer may comprise RNAse inhibitors to protect the RNA therein from degradation. Typically cDNA is prepared from the RNA sample using a reverse transcriptase enzyme and primers such as, oligo dT, random hexamers or gene specific primers.

The presence and/or level of one of the disclosed proteins can be determined using an isolated polynucleotide (e.g., a polynucleotide probe, an oligonucleotide probe/primer) capable of hybridizing to a nucleic acid sequence of one of the determinants described herein. Such a polynucleotide can be at any size, such as a short polynucleotide (e.g., of 15-200 bases), and intermediate polynucleotide (e.g., 200-2000 bases) or a long polynucleotide larger of 2000 bases.

The isolated polynucleotide probe used by the present invention can be any directly or indirectly labeled RNA molecule (e.g., RNA oligonucleotide, an in vitro transcribed RNA molecule), DNA molecule (e.g., oligonucleotide, cDNA molecule, genomic molecule) and/or an analogue thereof [e.g., peptide nucleic acid (PNA)] which is specific to the RNA transcript of the present invention.

Oligonucleotides designed according to the teachings of the present invention can be generated according to any oligonucleotide synthesis method known in the art such as enzymatic synthesis or solid phase synthesis. Equipment and reagents for executing solid-phase synthesis are commercially available from, for example, Applied Biosystems. Any other means for such synthesis may also be employed; the actual synthesis of the oligonucleotides is well within the capabilities of one skilled in the art and can be accomplished via established methodologies as known in the art utilizing solid phase chemistry, e.g. cyanoethyl phosphoramidite followed by deprotection, desalting and purification by for example, an automated trityl-on method or HPLC.

The above-described polynucleotides can be employed in a variety of transcript detection methods. A non-limiting list of RNA-based hybridization methods which can be used to detect the protein markers of the present invention include, Northern Blot analysis, Reverse-transcribed PCR (RT-PCR) analysis, RNA in situ hybridization stain

According to a specific embodiment, the polypeptides of some embodiments of the present invention are not used (or administered) as part of a regimen, which comprises treatment with D-penicillamine.

According to another embodiment, the polypeptides of some embodiments of the present invention are administered with immune modulating agents.

Examples of immune modulating agents include immunomodulatory cytokines, including but not limited to, IL-12, IL-2, IL-15, IL-7, IL-21, GM-CSF as well as any other cytokines that are capable of further enhancing immune responses; immunomodulatory antibodies, including but not limited to, anti-CTLA4, anti-CD40, anti-41BB, anti-OX40, anti-PD1, anti-PDL1 and chemokines such as CCL2, CXCL1 and CXCL10.

The polypeptide or polynucleotide encoding same of the present invention can be provided to the treated subject (i.e. mammal) per se (e.g., purified or directly as part of an expression system) or can be provided in a pharmaceutical composition comprising the polypeptide of the present invention. As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the polypeptide (or polynucleotide encoding same) of the present invention accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, PA, latest edition, which is incorporated herein by reference.

Suitable peripheral routes of administration may, for example, include oral, rectal, transmucosal, especially transnasal, intestinal or parenteral delivery, including intramuscular, subcutaneous and intravenous, intraperitoneal, intranasal, or intraocular injections.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water-based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water-based solution, before use.

The pharmaceutical composition of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients (nucleic acid construct) effective to prevent, alleviate or ameliorate symptoms of a disorder (e.g., ischemia) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

The Examples section which follows describes in details animal models for melanoma as well as standard tests to determine efficacy.

Dosage amount and interval may be adjusted individually to provide plasma or brain levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is affected or diminution of the disease state is achieved.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an U.S. Food and Drug Administration (FDA) approved kit, which may contain one or more-unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration (FDA) for prescription drugs or of an approved product insert. Compositions comprising a preparation of the invention formulated in a compatible pharmaceutical carrier may also be prepared, placed in an appropriate container, and labeled for treatment of an indicated condition, as if further detailed above.

As used herein the term “about” refers to +10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

Generally, the nomenclature used herein, and the laboratory procedures utilized in the present invention include molecular, biochemical, microbiological and recombinant DNA techniques. Such techniques are thoroughly explained in the literature.

### Materials and Methods

Cell culture: Ret-melanoma luciferase mCherry stable cells were grown in complete RPMI 1640 (with 10% FBS and 1% penicillin-streptomycin; Biological Industries) and 10 μg/ml puromycin (Sigma-Aldrich) for the selection of the stable cells. B16-F10 mouse melanoma cells were cultured in DMEM medium (with 10% FBS and 1% penicillin-streptomycin and 1% L-glutamine; Biological Industries). HDF cells were grown in DMEM (with 10% FBS and 1% penicillin-streptomycin; Biological Industries).

Cloning, expression and purification of AS1 and AS2: The human propeptide of LOX (22-168) was cloned into pYD5 mammalian expression vector and the plasmid was transfected into competent HEK 293-6E cells in exponential growth phase with polyethylenimine transfection reagent in a ratio DNA-PEI ratio of 1:2. After 120 h of incubation the cells were harvested and the supernatant was purified through a HiTrap Protein A column which was equilibrated with 100 mmol/L phosphate buffer (pH 8) and the elution was performed by 100 mmol/L citrate buffer (pH 4.5).

LOX and HSP70 expression and purification: The catalytic domain of human LOX (169-417) and HSP70 was cloned into pET28 expression vector and transformed into competent Escherichia coli BL21 SHuffle strain. Single colony was resuspended in 10 ml liquid medium with antibiotic. The 10 ml culture was used to inoculate 1 L of liquid medium. The culture was incubated at 30° C. until O.D₆₀₀ reaches 0.4-0.8 and 0.4 mM IPTG inducer was added. The culture was incubated overnight 16° C., 250 rpm. The medium was centrifuged for 15 min at 7800 g (Thermo LYNX 4000). The pelleted fractions were frozen for 30 min following incubation in ice with lysis buffer containing 50 mM Tris-HCl (pH 8), 200 mM NaCl, 40 mM Imidazole, lysozyme, protein inhibitor and DNase. The solution was sonicated and centrifuged at 59,889×g for 40 min (Thermo LYNX 4000). The suspension was purified with 0.22 um filter and loaded to a HisTrap column (GE Healthcare) which was pre-equilibrated with the following buffer: 50 mM Tris-HCl pH 8, 200 mM NaCl, 40 mM Imidazole. The protein was eluted with 50 mM Tris-HCl pH 8, 200 mM NaCl, 400 mM Imidazole. The enzyme was purified by size-exclusion chromatography using a HiLoad 16/60 Superdex 75 (Amersham Biosciences) and eluted with 50 mM Tris-HCl pH 8, 200 mM NaCl.

ELISA binding assay: A ninety-six-well plate (Nunc) was coated with LOX-catalytic domain, HSP70 or BSA at 10 μg/ml. After blocking with 2% BSA in PBS, the plate was incubated with the antibodies for 1 h at 37° C. Bound antibodies were detected by peroxidase-conjugated antibody goat anti-human (Jackson ImmunoResearch). EC₅₀ was calculated with GraphPad Prism from Find ECanything curve fitting analysis.

Differential scanning fluorimetry (DSF): The NanoDSF Prometheus NT.48 measures the Fc, AS1 and AS2 stability using intrinsic tryptophan or tyrosine fluorescence by continuously heating the samples with an adjustable heating rate of 1° C./min in a range of 20-95° C. and simultaneously reads both the fluorescence and back reflection signal. This approach is relying on the change of intrinsic tryptophan fluorescence at 330 nm and 350 nm. Protein unfolding leads to a change in the polarity around tryptophan residues, leading to a redshift of fluorescence.

Immunoprecipitation: AS1 and AS2 were covalently coated on magnetic protein G beads (Genescript) according to manufacturer's instructions. The coated beads were incubated with concentrated supernatant from 10 cm² dish culture of HDF cells overnight on 4° C. Pellet beads were obtained by centrifuge (˜500 g for 5 min 4° C.) and the beads were washed with PBS of volume at least 5 times the initial beads volume. Then the beads pellet was placed in a collection column, which was inserted in an eppendorf tube containing 10 ul of 1 M Tris-HCl pH 8. 90 ul of elution buffer (Thermo-Scientific) were added to the beads and after 5 min of incubation, elution was performed by centrifuge. The last step was repeated four times.

Glycosylation analysis of AS1 by mass-spectrometry: The samples were subjected to in-solution digestion, either by trypsin or with the alpha-lytic protease, followed by a desalting step. The resulting peptides were analyzed using nanoflow liquid chromatography (nanoAcquity) coupled to high resolution, high mass accuracy mass spectrometry (Fusion Lumos) in EThcD mode to allow the identification of the glycans. Each sample was analyzed separately in a random order in discovery mode. Raw data was processed using Byonic (by Protein Metrics) v3.3.11, using the human protein database and enabling the following modifications: Carbamidomethylation of C as fixed modification and mono-carbamidomethylation on HK/N-term (also enabled di-carbamidomethylation on N-term only), oxidation of MHW, deamidation on NQ and protein N-term acetylation as variable ones. A database of 132 know human glycans was used for the identification of the different glycosylations. Each of the identified spectra was manually validated.

Mass-spectrometry analysis of elutions after I.P with Fc, AS1, AS2: The samples were cluted using 5% SDS and subjected to tryptic digestion using an S-trap. The resulting peptides were analyzed using nanoflow liquid chromatography (nanoAcquity) coupled to high resolution, high mass accuracy mass spectrometry (Q Exactive HFX). Each sample was analyzed separately in a random order in discovery mode. Raw data was processed with MaxQuant v1.6.0.16. The data was searched using the Andromeda search engine against the human protcome database appended with common lab protein contaminants and the following modifications: Carbamidomethylation of C as a fixed modification and oxidation of M and protein N-term acetylation as variable ones. The LFQ intensities (label free quantification) were calculated and used for further calculations using Perseus v1.6.07. Decoy hits were filtered out, as well as proteins that were identified on the basis of a modified peptide only. The LFQ intensities were log transformed and only proteins that had at least 2 valid values in at least one experimental group were kept. GO annotations were added.

Two-photon microscopy and second harmonic generation: HDF cells were plated in 35 mm dishes in DMEM 10% FCS medium. Following the incubation in cell culture medium, the following factors were added in order to promote the extracellular matrix expression, 5 ng/ml EGF, 5 μg/ml insulin, and 150 μg/ml L-ascorbic, in the presence of Fc, AS1 and AS2 (5 μM). The ECM formation was monitored for 1 week with two-photon microscopy second harmonic generation. Images of the HDF and tissue samples were taken using 2 PM: Zeiss LSM 510 META NLO microscope equipped with a broadband Mai Tai-HP-femtosecond single box tunable Ti-sapphire oscillator, with automated broadband wavelength tuning 700 to 1,020 nm from Spectra-Physics using an 855-nm wavelength (detection at 400 nm) and a 20× objective.

Image analysis for directionality and entropy: Imaging analysis was done using Fiji package. Fourier component analysis for directionality was performed on data using the Fiji plug-in “Directionality” created by Jean-Yves Tinevez (www (dot) pacific (dot) mpi-cbg (dot) de/wiki/index (dot) php/Directionality) and following Fiji's instructions. For entropy calculation the Fiji macro CDS.ijm was used for the creation of orientation analysis files and afterwards mean entropy was calculated by matlab script developed by O. Golani & G. Molodij and modified by E. Shimshoni. Thickness was calculated based on the Z-stack size of collagen fibers.

Histological staining: The slides were exposed to Hoechst stain (1:4,000; Invitrogen Probes) for 1 min. Sirius Red staining was performed to label Collagen type I and III, and Reticulin staining for Collagen type III. Masson's trichrome was performed to stain collagen, cytoplasm and nuclei (dark brown). For microscopic analysis, a Nikon light microscope (Eclipse E800) equipped with a Nikon digital camera (DS-Ril) was used.

Mice: All animal experiments were performed in accordance with guidelines of the Tel Aviv University Institutional Animal Care and Use Committee with institutional policies and approved protocols (IACUC permit: 01-20-033). Male wild-type C57BL/6J mice (10-12 weeks old) were purchased from Envigo and maintained in the Tel-Aviv University animal house as per the Institutional Animal Care and Use Committee protocols for all the experiments.

### Melanoma Injections in Mice:

Intravenous injection: Mice were injected with 0.5×10⁶ Ret-melanoma luciferase mCherry stable cells suspended in 100 ul of sterile PBS (without Ca²⁺ and Mg²⁺) and were injected i.v. in the lateral caudal vein of the mouse tail for the indicated experiment.

Subcutaneous injection: Mice were injected with 0.2×10⁶ Ret-melanoma luciferase mCherry stable cells suspended in 50 ul of complete RPMI 1640 (with 10% FBS and 1% Pen/strep; Biological Industries) and were injected s.c. above the right flank for the indicated experiment.

Drug treatments: Fc, AS1 or AS2 in the dose of (5 mg/kg bodyweight) were injected i.v. in the lateral caudal vein of the mouse-tail every other day (alternate day injection following the melanoma injection) for the indicated experiments.

Immunohistochemistry Paraffin (IHC-P): Human tissue samples from different stages (compound nevi, in situ melanoma, lymph metastasis and liver metastasis) were procured from Tel Aviv Sourasky medical center BioBanks (Helsinki ethical approval 16-660-TLV-7) and the Wolfson Medical Center (Helsinki ethical approval WOMC 0039-18). Tissues were fixed with 4% paraformaldehyde at 4° C., dehydrated in a graded ethanol series, and embedded in paraffin wax. The tissue slides were de-paraffinized in xylene, hydrated in a graded series of ethanol, and subjected to a microwave sodium citrate buffer (pH=6.0) for antigen retrieval. Tissue samples were blocked with 5% BSA, 0.5% Tween-20 in PBS, and then incubated with HMB45 (ab732, Abcam), S100 beta (ab52642, Abcam), Hsp70 (ab2787, Abcam) and LOX (ab174316, Abcam) antibodies, followed by an incubation with the associated fluorophore-conjugated secondary antibodies: Alexa Fluor 488 (ab150105, Abcam), Alexa Fluor 488 (A11008, Invitrogen), Alexa Fluor 594 (A21203, Invitrogen), and Alexa Fluor 594 (A21207, Invitrogen). Nuclear staining was performed with DAPI (Vector Laboratories). Images were obtained at ×40 magnification using fluorescence microscopy (Nikon).

Immunofluorescence analysis of human specimens: Immunofluorescence analysis. For intensity quantification of the IHC-P, images (HSP70+S100, and LOX+HMB45 combinations) were captured and the (non-nevi and nevi) or (stoma and tumor) regions were marked in ×40 images; at least ≥15 areas were quantified per image (stroma or tumor), taken from at least three different tissue samples following the above-noted in-vivo experiments. Fluorescence images with ×40 magnification were split into separate channels and converted into 8-bit images using ImageJ software. A specific area in each image (8-bit) was subjected to quantification using the ROI manager function, to precisely quantify the intensity from the same place in different channels simultaneously; each intensity was normalized to DAPI from the same image to rule out discrepancies due to differences in cell numbers. The graph represents the mean fluorescence intensity (marker/DAPI) for each target in the stroma and tumor regions.

Immunofluorescence staining of mice tissues and human tissue array: Samples were fixed with PBS 4% PFA, paraffin embedded and sectioned (4 μm). The Formalin-fixed paraffin-embedded (FFPE) slides were de-paraffinized using xylene for 10 min (×2), ethanol 100% for 10 min, ethanol 96% for 10 min, ethanol 70% for 10 min and PBS for 5 (×3). Antigen retrieval was performed in 0.1M Citric Acid, pH 6.0 using microwave until boiling and 15 min on 20% intensity, followed by washes with PBS. Samples were blocked in PBS, 20% normal horse serum, and 0.2% Triton X-100 (20 min, 25° C.) and then incubated with primary Ab in PBS, containing 2% normal horse serum and 0.2% Triton X-100 (60 min, 25° C.). Continuing, samples were washed three times in PBS and incubated with a secondary antibody (60 min, 25° C.) and mounted in a mounting medium. Primary Abs: anti HSP70 (ab2787, Abcam), anti-LOX (ab174316). For the tissue array 1 ug/ul biotinylated-AS1 and biotinylated-AS2 were used as primary antibodies. Secondary antibodies were used Streptavidin-Alexa 488, anti-mouse Alexa 647, anti-Rabbit Alexa 647

Quantification of tumor binding by AS1 and AS2 in FFPE samples: Stained slides were scanned using a 3D Histech Pannoramic SCAN II slide scanner. Post processing of the scanned raw data included identification of all nuclei in the sample using QuPath v0.2.0-m9 cell detection function. Threshold parameters were optimized to identify nuclei of all shapes and sizes, and AS1 and AS2 signal was quantified within the nuclear border. To negate potential bias, the training was performed on the basis of a multitude of parameters derived from the nuclear data layer (DAPI) only. The software could then distinguish between AS1 and AS2 positive and negative cells and calculate the percentage of AS1- and AS2-positive cells within each compartment. Quantification of LOX and HSP70 populations was performed in a similar manner using samples stained for anti-LOX and anti-HSP70.

In vivo luciferase experiment: Male wild-type C57BL/6J mice (10-12 weeks old) were obtained from Envigo. Institutional Animal Care and Use Committee approved all experimental protocols (IACUC permit: 01-20-033). Ret-melanoma luciferase mCherry stable cells were transfected with the indicated miRNA mimic or control three times in the in vitro culture and 0.2×10⁶ cells were injected i.v. Following the injections, mice were injected every other day (3 weeks) with Fc, AS1 and AS2 (dose=5 mg/kg). Mice were injected with 150 ul of D-luciferin (15 mg/ml) (Biovision) and imaged weekly using the IVIS® Spectrum in vivo imaging system (Perkin Elmer) to track the weekly status of invasion in vivo for 3 weeks. Images were quantified for the intensity of the luminescence as Avg Radiance [p/s/cm²/sr].

Ex vivo luciferase experiment: The mice were injected were injected (i.p.) with 150 ul of D-luciferin (15 mg/ml) and anesthetized using ketamine/Xylazine and lungs were dissected for ex-vivo luciferase imaging. Images were quantified for the intensity of the luminescence as Avg Radiance [p/s/cm²/sr].

FACS analysis: Male wild-type C57BL/6J mice (10-12 weeks old) following the melanoma cells injection and drug treatments (as mentioned above) were anesthetized using ketamine/Xylazine and cardiac punctured as described previously PMID: 21350616. Blood was immediately collected in EDTA-coated tubes and stored on ice followed by Red blood cells lysis (final concentration of 150 μM NH₄Cl, 10 μM NaHCO₃ and 1.26 μM EDTA in ddw) for 10 min at room temperature followed by neutralization with cold PBS (×1) and centrifugation at 800×g for 5 min at 4° C. The cell pellet was re-constituted in FACS buffer (0.5% FBS and 2 mM EDTA in PBS) with DAPI (vector labs) and subjected to FACS analysis for mCherry and DAPI signal to detect the mCherry⁺DAPI⁺ cells from the samples.

Scratch Assay: B16-F10 cells were seeded in a 6 well plate at a density of 1.5*10⁶ and upon 95-100% confluence the monolayer of cells was wounded using a plastic pipette tip to create a scratch of 450 μm diameter approximately. The cells were afterwards treated with 1 μM of the FC, AS1 and AS2 drug respectively and incubated at 37° C. with 5% CO₂ and monitored on the IncuCyte® Live Cell Analysis System (Sartorius, USA). Total of 5 images of the wounded area per condition were taken at 0 h, 6 h, 12 h, 18 h, and 24 h and analyzed using ImageJ software (RRID: SCR_003070). Percentage migration area was calculated by dividing the healed area by the wounded area.

Invasion Assay: WM3314, WM1716, B16-F10 and Ret-melanoma cells were seeded in a 12 well plate at a density of 0.5*10⁶ and treated with 1 μM and 10 μM of the Fc, AS1 and AS2 drugs respectively for 24 hours. The cells were then trypsinized and seeded at a density of 5*10⁴ in serum-free DMEM for 9 hours into a transwell chamber (Corning) with a membrane of 8 μm pore size coated with matrigel (BD Bioscience). The bottom of the chamber contained DMEM with 10% FBS, which served as an attractant. Cells were fixed using methanol for 20 minutes at room temperature. Nuclei were labeled with 4′,6-diamidino-2-phenylindole (DAPI; Vector Laboratories) and images were taken using an Olympus IX81 microscope and the cellSens dimension software. Three fields per well were photographed and the percentage of invaded cells compared to the number of total non-invaded cells per treatment respectively was measured.

### In Vitro Melanoma T Cell Kill Assay

Mice injection and tumor injection: Mice were injected with 0.2×10⁶ Ret-melanoma luciferase mCherry stable cells were suspended in 50 ul of complete RPMI 1640 (with 10% FBS and 1% Pen/strep; Biological Industries) and were injected s.c. above the right flank and sacrificed at day 8.

T cell isolation: For isolation of T cells from splenocytes, spleen from mice (with tumor inoculation) was excised in HBSS and strained using 70 μM cell strainer (Corning, USA) in 50 ml conical tubes. Cells were then washed by centrifugation at 800 g for 5 min at 4° C. The cells were treated for the Red blood cells lysis (0.15 M NH₄Cl, 0.01 M NaHCO₃ and 1.2 mM EDTA in ddw) for 10 min at room temperature followed by neutralization with cold PBS (×1) and centrifugation at 800 g for 5 min at 4° C. Cell pellet was carefully re-suspended in 5 ml Complete RPMI 1640 followed by carefully adding 5 ml of Histopaque®-1077 (Sigma Aldrich) at the bottom of the tubes in 1:1 ratio and carefully pulling out the pipette without disturbing the layers. Cells were centrifuged at 400 g for 30 min at room temperature with acceleration “0” and deceleration “0”. After centrifugation, the opaque layer at the interface was carefully collected containing mononuclear cells using pasteur pipette into a clean conical centrifuge tube. The cells were washed by adding 10 mL of complete RPMI 1640 medium and mixed by gently drawing in and out of a Pasteur pipette. The cells were centrifuged at 250 g for 10 min at 4° C. and the pellet was re-suspended in MACS buffer (0.5% BSA, 2 mM EDTA in PBS (×1)). For CD8 enrichment, 10 ul CD8 mouse MicroBeads (Miltenyi Biotech) were used for 10×10⁶ cells were used and incubated for 15 min at 4° C. The LS Columns (Miltenyi Biotech) were prepared by passing 500-ul MACS buffer and discarding it. 500-μl MACS buffer was added to the samples allowing it to pass through it and collected the flow-through in a 15 ml conical tube. This step was repeated again. The columns were washed with 2 ml of MACS buffer to get rid of the unbound cells and this step was repeated two times. The columns were placed in new 15 ml conical tubes and 2 ml MACS buffer was injected into the columns for eluting the CD8⁺ cells.

T cell culture and expansion: T cells were cultured in RPMI-1640 supplemented with 1% penicillin-streptomycin, 10% heat-inactivated FBS, 1% sodium pyruvate (Sigma-Aldrich), 1% MEM-Eagle non-essential amino acids (Biological Industries), 1% insulin-transferrin-selenium (Sigma-Aldrich), and 50 μM β-mercaptoethanol (Sigma-Aldrich). For T cell expansion, culture dishes were pre-coated with 0.5 μg/mL anti-CD3 (clone 17A2) and 0.5 μg/mL anti-CD28 (clone 37.51) LEAF antibodies (both purchased from BioLegend) in PBS and were supplemented with 1,000 IU/mL recombinant murine IL-2 (PeproTech) overnight.

Melanoma T cell co-culture: 0.001×10⁶ Ret-melanoma luciferase mCherry cells were plated in 96 well plate overnight and/or co-cultured with 0.006×10⁶ activated T cells at ratio (1:6) with indicated drug treatments with or without anti-PD1 (CD279, clone #RMP1-14) treatments. The cells were incubated at 37° C. with 5% CO₂ and monitored on the IncuCyte® Live Cell Analysis System (Sartorius, USA).

Image analysis: The image analysis was performed for day 0, 2 and 4 following the co-culture experiment. Four images were taken at random places from each well at each time-point and these imaging places were automatically kept constant throughout the timeline. A random image was snip-extracted from the IncuCyte S3 2019A software for each time point and was converted to the 32-bit image. The number of melanoma cells were counted using PHANTAST tool in ImageJ software which gives the confluence of cells/area. The tool takes into consideration the cell shape, convexity and size, which helped us to exclude the T cells which are significantly smaller than the Ret-melanoma cells used in the study. For each time-point Ret melanoma/T cells co-culture were normalized to the Ret-melanoma cells.

RNA sequencing: Lungs from animals perfused with ice cold PBS and flash freezed in liquid nitrogen. RNA was extracted from melanoma tumors of mice lungs using TRIzol® Reagent and miRNeasy® Mini Kit (Qiagen) following the manufacturer's protocol. The concentration and quality of extracted RNA were measured using NanoDrop® and analysed by Agilent 2200 TapeStation system. For library preparation the Bulk-MARS sequence protocol was used in which the 3′UTR of the transcripts were captured. The libraries were sequenced using illumine NovaSeq 6000. In bioinformatics analysis the number of the reads for each gene in each sample was counted using HTSeq-count. Data normalization and differential expression analysis was calculated using the DESeq2 package.

Enrichment pathway analysis: Enrichment analyses for RNA-seq dataset were performed using Metascape, a powerful web-based tool, which involved in four processes: ID Conversion, Gene Annotation, Membership Analysis, and Enrichment Analysis. The available terms for enrichment analysis include pathway (Reactome Gene Sets, Canonical Pathways, BioCarta Gene Sets, GO Biological Processes, Hallmark Gene Sets, and KEGG Pathway), functional set (Go Molecular Functions), structural complex (Go Cellular Components, KEGG Structural Complex, and CORUM Protein Complex), and signature module (immunologic signatures, oncogenic signatures, and chemical and genetic perturbations). We undertook enrichment analysis for AS1 compared to Fc expressed genes and AS2 compared to Fc in two comparison cohorts independently. The used values had a cut-off criterion Log|FC|>0.6 for AS1 vs Fc and Log|FC|>0.8 for AS2 vs Fc.

Further enrichment analysis was performed using STRING version 11.0 which offers a genome-scale input, with each gene having an associated numerical value (a measurement or statistical metric); in our case we used log 2Fc values. The computing of functional enrichments within each functional classification framework (GO, KEGG, InterPro, etc.) was performed based on developers' instructions.

Mass cytometry and cell sorting: Cell isolation of lung samples was performed as previously described. In brief, lungs were harvested at the end of the experiment from Fc-treated mice, AS1-treated mice, and AS2-treated mice, were carefully separated from the surrounding tissues and cleaned with cold PBS. Then, the whole lung tissue was minced and immersed into RPMI-1640 medium (GIBCO, 21875034) containing 10% FBS, 2 mM HEPES, 0.05 mg DNase (Roche, 04716728001), and 0.5 mg/mL of collagenase VIII (Sigma-Aldrich, C2139) and placed for 40 min at 250 rpm shaking at 37° C. and mashed through a 70 μm cell strainer.

Following, cells were stained according to a previously published protocol. Individual mice cell suspensions were stained with Cell-ID Cisplatin 0.125 μM for viability and fixed using Maxpar® Fix I Buffer. Samples were then permeabilized using Maxpar® Barcode Perm Buffer and then barcoded using The Cell-ID™ 20-Plex Pd Barcoding Kit, allowing us to merge samples for antigen staining. Before analyzing, the cell suspension was incubated with Cell-ID Interculator Iridium for 20 min.

Cells were analyzed with a cyTOF2® mass cytometer (Fluidigm). Results were normalized and debarcoded using Fluidigm cyTOF software. CyTOF results gating and further analysis was done with FlowJo software (FlowJo, LLC) and cell populations were defined according to the named markers. tSNE analysis was done using the viSNE application in the Cytobank web platform. tSNE (t-Distributed Stochastic Neighbor Embedding) is a machine-learning algorithm used to cluster multivariate data into a 2D representation.

Statistical analyses: Data were analyzed by unpaired, two-tailed 1-test to compare between two groups or by one-way ANOVA to compare several groups. All analyses were performed at least triplicate. Statistical analysis was performed using GraphPad Prism 7. Data are presented as mean±SEM; values of p<0.05 were considered statistically significant (*P<0.05, **P<0.01, ***P<0.001).

### Results

### LOX and HSP70 are Biomarkers of Human Melanoma Cancer Progression

LOX and HSP70 are major regulators of collagen production and assembly and organization, pivotal processes for the development of tumor-supporting microenvironment. To evaluate their relevance as biomarkers of human melanoma the present inventors analyzed the overall survival (OS) rate of melanoma patients as a function of alterations in LOX or HSP70 levels. The median months OS (95% CI) rates were significantly lower in patients with altered LOX levels 24.53 (20.35—NA) than in those with unaltered LOX levels 61.10 (51.90-66.67) (FIG. 1A). A similar trend was observed in patients with altered HSP70 levels, who had significantly shorter survival rates 34.75 (23.92-94.60) than those with unaltered HSP70 levels 65.44 (60.18-71.75) (FIG. 1B). Further clinical analysis of normal and melanoma human specimens confirmed that LOX and HSP70 levels were increased in melanocytic nevi and melanoma tissues (Figures IC, ID, respectively). The study of LOX and HSP70 deposition in different sites and at different stages of human melanoma, demonstrated a significant increase in melanocytic nevi tissues, which further increased during disease progression (FIG. 1E). Approximately 25-33% of cutaneous melanomas arise from nevi after accumulation of genetic and epigenetic changes, even though the vast majority will never progress to melanoma. Upon the linear progression theory of normal melanocytes transformation to melanoma, the different steps are formation of banal nevi, dysplastic nevi, melanoma in situ, and ultimately invasive melanoma. At the stage of in situ melanoma LOX level was increased by 50% compared to nevi tissues, while the HSP70 level was increased by 100% compared to nevi (FIG. 1F). In advanced melanoma and specifically in lymph node metastasis, LOX and HSP70 levels were increased by 4-fold and 3-fold respectively, compared to nevi tissues (FIG. 1G). Finally, in liver melanoma metastasis, the levels of LOX and HSP70 were similar to those found in in situ melanoma (FIG. 1H). Their expression levels were increased in primary melanoma sites, but were further increased in lymph node melanoma metastasis showing that LOX and HSP70 are biomarkers of human metastatic melanoma. These findings provided the motivation to engineer a decoy receptor combining the inhibition of both LOX and HSP70, in order to halt melanoma disease progression by modulating the tumor microenvironment.

### Design and Characterization of Fc-Fused Bispecific LOX/HSP70 Decoy Receptors

The recombinant propeptide of lysyl oxidase (LOX-PP) binds to HSP70 and ECM-associated molecules including the degradation and cross-linking enzymes MMP-2 and LOXL2 as part of its tumor-suppression effect, and both LOX and HSP70 are highly expressed in human metastatic melanoma as shown above. The LOX-PP/HSP70 interaction has been mapped to the HSP70 peptide-binding domain and to LOX-PP amino acids 26 to 100. Thus, it was hypothesized that LOX-PP, in addition to HSP70 targeting, might also bind to the processed catalytic domain of LOX and inhibit its activity, and LOX-PP-based bispecific decoys were designed. Since LOX-PP is a very flexible and disordered molecule, a fusion of LOX-PP was synthesized with the crystallizable (Fc) domain of a human IgG1 antibody (FIG. 2A) to increase its stability. There are three glycosylation sites (N81, N97 and N144) in the LOX-PP sequence. We generated the fused protein in a glycosylated form (AS1) and a non-glycosylated (AS2) form (FIG. 9A and Methods).

Both AS1/AS2 decoys were engineered as dimers stabilized by disulfide bonds in the hinge region of the Fc fragments. This dimerization created antibody-like molecules of approximately ˜84 kDa that were significantly smaller than a monoclonal antibody of the same isotype (˜150 kDa). The stability of AS1 and AS2 was measured by differential scanning fluorimetry (DSF). The unfolding transition temperatures (Tm) were 69.9° C. for Fc, 70° C. for AS1 and 69.5° C. for AS2 (FIG. 2B). AS1/AS2 were thus stable up to this temperature, and had a similar stability, showing that the glycosylation did not contribute to the stability of the inhibitor. Both inhibitors showed two unfolding transitions when the fluorescence ratio (F330/F350) was plotted as a function of temperature and the first derivative of the unfolding curves (FIGS. 2A and 2B). The distinct unfolding events can be attributed to the different thermal stabilities of the Fc-domain of the inhibitor.

The affinity of AS1 and AS2 for the recombinant catalytic domain of LOX, estimated by the EC₅₀ value, was 43±1.2 nM and 58±1.2 nM respectively (FIG. 2C), whereas their affinity for recombinant HSP70 was 41±1.2 nM and 57±1.2 nM respectively (FIG. 2D). Both AS1 and AS2 interacted in vitro recombinant LOX and HSP70 with similar and high affinity, although the EC₅₀ values were somewhat higher for AS2. The lack of glycosylation of the LOX-PP moiety did not alter the binding demonstrating that the glycosylation is not required for the binding of AS1 and AS2 to LOX and HSP70.

Next, the interactions of AS1 and AS2 with endogenous LOX and HSP70 proteins from melanoma cell lines were assessed by co-immunoprecipitation. Beads pre-coated with AS1 or AS2 decoys and Fc as control were incubated with culture media of Ret melanoma cell line. In Ret-melanoma cells culture media, LOX and HSP70 were both precipitated from Ret-melanoma cells culture media by AS2 whereas only LOX was precipitated in detectable amount by AS1, in apparently higher amount than by AS2 (FIG. 2E). The unglycosylated form of the inhibitor is thus able to precipitate both LOX and HSP70 from this melanoma cell line whereas the glycosylated form precipitates only LOX. In addition, LOX was immunoprecipitated from culture media of human dermal fibroblasts (HDFs) by AS2 but the amount immunoprecipitated by AS1 was similar to the amount immunoprecipitated by the control (Fc) (FIG. 2H). HSP70 immunoprecipitation in culture medium of HDFs by AS1 and AS2 detected by proteomic analysis. The proteins strongly adsorbed on the AS1 and AS2 beads revealed the presence of HSPA6/7 (corresponding to heat shock 70 kDa protein-6/-7) and of HSPA1A/B (Heat shock 70 kDa protein 1A/1B/2) showing that HSP70 can be immunoprecipitated by both AS1 and AS2 and that is was more tightly bound to the beads than LOX (FIGS. 2F, 2G, FIG. 9B).

These results showed that LOX-PP-based decoys can effectively bind both the catalytic domain of LOX and HSP70. Then, the present inventors tested if they were able to inhibit the functional activities of LOX and HSP70 in Ret melanoma cells and HDFs, which overexpress ECM proteins, mostly collagen, with a key role for LOX. HDFs were cultured with AS1, AS2 or control Fc, and collagen fiber packing, distribution and organization, determined by second harmonic generation, were found to be altered in presence of AS1 and AS2 (FIG. 2I). Measuring the entropy, it was found that both AS1 and AS2-treated cells present disorganized and non-parallel collagen fiber organization as shown by the increase in entropy (i.e., alignment) of collagen fibers), which reached statistical significance for AS2 compared to the Fc control (FIG. 2J). Additionally, AS1/AS2-treated HDF cells inhibited LOX activity by significantly reduced the thickness of deposited collagen compared to control with a more pronounced effect for AS2 (FIG. 2K). These results show that AS1, and AS2 to a higher extent, interfere with collagen fiber assembly by disrupting their alignment and decreasing their diameters.

Next, the ability of AS1, AS2 and Fc as a control was evaluated to inhibit the ATPase activity of HSP70. The ATPase activity was reduced in both the culture media and in the lysates of Ret-melanoma cells treated with AS1 and AS2 compared to Fc (FIGS. 2L and 2M, respectively). However, only the decrease observed in the culture media was statistically (AS1-30%, AS2-18%) significant. Both decoys might be more efficient to inhibit the activity of the extracellular HSP70 (cHSP70) than the intracellular ones because the size of AS1 and AS2 might prevent their internalization. Both bispecific decoys alter collagen fibrillogenesis and the organization of collagen fibers as a consequence of their binding to LOX and inhibit the ATPase activity of HSP70, thus acting on both targets as hypothesized.

### AS1 and AS2 Inhibit Melanoma Cells Invasion and Migration In Vitro

Both AS1 and AS2 significantly inhibited the invasiveness of two highly metastatic human melanoma cell lines (˜3.5-fold, FIG. 3A), and two murine cell lines B16-F10 (˜6-fold, FIG. 3B), and Ret-melanoma cell line invasion (˜3-4-fold, FIG. 3C). In wound healing assays performed with B16-F10 melanoma cell, the percentage of the cells in the migrated (scratched) area decreased as a function of time, with a more pronounced decrease for control cells than for cells treated with AS1 and AS2 (85%, FIG. 3E) suggesting that AS1 and AS2 inhibited melanoma cell migration. AS1 and AS2 induced a decrease in N-cadherin, a marker of cell-cell adhesion, 68% and 49%, respectively, and in vimentin, a marker of epithelial-mesenchymal transition (EMT) in a melanoma cell line, 52% and 59&, respectively (FIG. 3F, 3G, 3H, 3I). These data show that AS1 and AS2 inhibit migration and invasion of melanoma cells, and that this effect is mediated by their regulation of LOX and HSP70 activity in the tumor microenvironment, and on EMT.

### Bispecific LOX/HSP70 Inhibition Prevents Melanoma Metastasis to the Lungs

The impact of AS1 and AS2 decoys on melanoma disease progression was then tested in vivo, injecting stable luciferase expressing Ret-melanoma cells in C57BL/6J mice³¹ (FIG. 4A). By the second week of treatment, significantly lower levels of metastatic melanoma cells in the lungs of AS1- and AS2-treated mice were observed (FIGS. 4B, 4C). After the third week of treatment ex vivo lung bioluminescence imaging was performed (FIG. 4D). The number of metastatic melanoma cells in lung tissue was significant reduced in AS1- and AS2-treated mice (FIG. 4E). Moreover, no circulating melanoma cells were detected by flow cytometry in the blood of AS1- and AS2-treated mice in contrast to Fc-treated mice (FIGS. 4F and 4G). Histological analysis of lung tissue sections ex vivo showed that the tumor area was significantly smaller in AS1- and AS2-treated mice than in Fc-treated mice (FIG. 4H). Labeling LOX and HSP70 in lung tissues from non-treated mice provided information on the location where both molecules are expressed (FIG. 10A). LOX was mainly expressed in metastatic melanoma cells in the tumor area of the lungs and around bronchi in the healthy area high levels of HSP70 were found in the tumor area but HSP70 was also distributed in the entire lung including alveoli in healthy areas (FIG. 10A). In summary, LOX and HSP70 are overexpressed in melanoma tumors and to a lesser extent in healthy lung regions of melanoma-bearing mice.

To examine whether AS1 and AS2 physically reach and recognize LOX, paraffin sections from metastatic lung tissue were stained after treatment with biotinylated anti-human Fc antibody, which recognize Fc region of AS1 and AS2, and commercial anti-LOX antibody. Imaging of lung tumor area, produced strong immunofluorescence signal verifying that both inhibitors are localized at the lung tumor and co-localized with commercial anti-LOX antibody (FIG. 10B). Moreover, the signal indicating their binding is robustly detected in melanoma metastatic tissue and is much weaker in healthy lung tissue (FIG. 10B, lower).

### AS1/AS2 Decoys Modulate Immune Cells in the Tumor Microenvironment

Initially, LOX and HSP70 expression by different cell types of the lung tissue of metastatic melanoma in Fc treated mice was compared. As expected, LOX and HSP70 were overexpressed in melanoma cells, however a significant amount of LOX was also expressed in neutrophils, dendritic cells (DCs) and macrophages, and HSP70 was highly expressed in macrophages (FIG. 5A). Interestingly, LOX/HSP70 co-expression was detected in the majority of melanoma cells, and at lower levels in DCs and macrophages (FIG. 5B). To determine if AS1 and AS2 modulate immune cells, mass-cytometry analysis was performed on whole lung extracts from treated mice (FIG. 5C) to map a wide range of immune cells, ret-melanoma cells (anti-mCherry), endothelial and epithelial cells, HSP70 and LOX. AS2, but not AS1, significantly reduced melanoma cell proliferation (FIG. 5D, 5E). Evaluation of the in vivo treatment showed that AS2 effectively reduced the percentage of melanoma cells including the LOX+/HSP70+ cells (FIG. 5F). In addition, AS2 had a stronger reducing effect than AS1 on the frequency of immune cells (FIGS. 5G-I). The role of CD4⁺/CD8⁺ T-cells in melanoma progression is still unclear. Recent evidences show that CD4⁺/CD8⁺ T-cells, which infiltrate the tumor microenvironment, arise from CD8⁺ T-cells that are reprogrammed to a T helper function. Here, a significant reduction in the percentage of CD4⁺/CD8⁺ T-cells in the lungs of AS2-treated mice was observed (FIGS. 5K, 5L), without affecting CD8⁺ T cells (FIG. 5J), supporting a microenvironment-mediated effect. Key players were the antigen-presenting cells DCs and macrophages. Indeed, AS2 significantly reduced DCs frequency. This reduction could be triggered either by direct decoy targeting or indirectly, by elimination of LOX/HSP70 in the tumor microenvironment (FIGS. 5M, 5N). Finally, we found that AS2 had a strong inhibitory effect on macrophages (FIGS. 5O, 5P). In summary, AS2 not only inhibits melanoma tumor growth and metastasis formation, but plays also a role in regulating immune cells whereas AS1 was less efficient.

To further investigate the effects of AS1 and AS2 on immune cell activity, a T-cell killing assay was established. Since treatment with immune checkpoint inhibitors is the principal current therapy of metastatic melanoma, the effect of AS1 and AS2 was tested in combination with an anti-PD1 antibody, which blocks the immune checkpoint interaction of PD-1 with its cognate receptors eliciting a strong immune response. CD8+ T cells isolated from spleens of tumor-inoculated mice were co-cultured with Ret-melanoma cells and treated with AS1/anti-PD1 or with AS2/anti-PD1. The combination of AS1 or AS2 with the anti-PD1 enhanced the killing of melanoma cells compared to treatment with AS1, AS2 or anti-PD1 alone with a more pronounced effect of the AS1-anti-PD1 combination (FIGS. 5Q, 5R). It should be noted that the combination of AS1 and anti-PD1, but not the combination of AS2-anti-PD1, were statistically more efficient that the Fc-anti-PD1 combination (FIG. 5Q). These results suggest that AS1, and to a lesser extent AS2, impact melanoma cells in vivo directly, but also indirectly by modulating the T cell differentiation programs towards activation of CD-8⁺ T cell-mediated killing.

### Decoy Treatment Suppresses Migration and ECM-Remodeling Pathways in Metastatic Melanoma

To determine if AS1 and AS2 modulate transcription programs of metastatic melanoma, melanoma cells were isolated from the lungs (FIG. 6A) for RNA-sequencing. One hundred genes involved in migration and differentiation were then selected (FIG. 6B) and submitted to enrichment analyses using Metascape (FIGS. 11A-B). Both AS1 and AS2 induced the downregulation of numerous genes, more genes being down-regulated in AS2-treated mice (FIG. 6B). Both AS1 and AS2 down-regulated genes involved in the regulation of cell adhesion, cell junction organization, negative regulation of cell population proliferation, negative regulation of locomotion, epithelial differentiation, and transmembrane receptor protein tyrosine kinase pathway but also tissue morphogenesis and heart development (FIGS. 11A-B). Their common targets also included genes involved in vasculature and blood development. By downregulating pathways controlling cell adhesion, proliferation and locomotion, AS1 and AS2 contribute to decrease melanoma aggressiveness and metastasis. AS1 specifically downregulated the expression of genes involved in actin cytoskeleton organization, regulation of ion transport, muscle contraction and pattern specification process (FIG. 11A) whereas AS2 specifically down-regulated genes involved in the positive regulation of cell motility, response to wounding, tissue migration, and blood circulation but also supramolecular fiber organization, external encapsulating structure organization and skeletal development (FIG. 11B).

Further analysis using KEEG annotations showed that both AS1/AS2 downregulated ECM-receptor interactions, focal adhesion, axon guidance, and Rap1 signaling pathway (FIGS. 6C-D), whereas AS1 specifically down-regulated the Ras signaling pathway (FIG. 6C), and AS2 the TGFβ and calcium signaling pathways (FIG. 6D). Additional enriched pathways associated with genes down-regulated by both AS1 and AS2 were identified using Reactome. They included ECM-related pathways (collagen formation, assembly of collagen fibrils and other multimeric structures, ECM proteoglycans), ECM interactions (laminin interactions and integrin cell surface interactions) and MET-related pathways (MET promotes cell signaling and activates PTK2 signaling) (FIGS. 6E, 6F). AS2 specifically down-regulated genes associated with the ECM [cross-linking of collagen fibrils (Col4a1/2/3/4/5/6, Col1a1, Lox), anchoring fibril formation-anchoring fibrils are made of collagen VII which anchor the dermis to the epidermis and ‘anchoring plaques’ are composed of type IV collagen and laminin 332 (Lamc2, Col4, Lama3, Lamb3) and collagen degradation] and with neutrophils (FMO oxidizes neutrophils-metabolites of drugs) (FIG. 6F). These computational analyses confirmed that both AS1 and AS2 inhibit cell proliferation and migration via suppression of MET signaling pathway by impacting ECM organization with a focus on collagens and laminins, and for AS2 on cross-linking of collagen fibrils and ECM-cell interactions mediated by integrins.

### AS2 Suppresses Pro-Metastatic ECM-Remodeling Enzymes and Expression of EMT Marker

Given their role of ECM-modulation in metastasis, the present inventors analyzed the expression of certain pro-metastatic ECM-remodeling enzymes, namely MMP-2, MMP-7 and ADAM17 which are implicated in melanoma growth and metastasis and were upregulated in melanoma cells (FIG. 9C), macrophages and T cells. AS2 significantly downregulated MMP-2 expression in melanoma cells (FIG. 7A), ADAM-17, MMP-2 and MMP-7 in macrophages (FIG. 7B) and dendritic cells (FIG. 7D), and ADAM-17 and MMP-7 in T-cells (FIG. 7C) in keeping with its downregulation of the pathway associated with collagen degradation identified above. Intensive ECM-remodeling being associated with EMT, the present inventors analyzed the levels of the EMT-associated oncogene FAM3C/ILEI, which is overexpressed in melanoma and other cancers. Both AS1 and AS2 strongly decreased the protein expression of FAM3C/ILEI in metastatic melanoma cells isolated from melanoma lungs metastasis (FIG. 7E).

AS1 and AS2 bind LOX⁺/HSP70⁺ expressing tumors in other malignancies

To check if the therapeutic approach mediated by AS1 and AS2 could be used for the treatment of any LOX+/HSP70+ tumors, a tissue array from 24 human biopsies (26 tumor types in FIGS. 8A-B) including samples from 500 patients from 20 different human organs, and studied the binding of AS1 and AS2 to these samples. AS1 and AS2 bound to all tumor types (FIGS. 8A and 8B). Among the major targets were prostate adenocarcinoma, testis embryonal carcinoma, ovarian cancer, uterus carcinoma, pancreatic adenocarcinoma, astrocytoma and glioblastoma (FIGS. 8A, 8B). The binding levels were likely highly related to the increased expression levels of LOX and HSP70 in these tumor types. The tumor tissues were immunostained with specific anti-LOX and anti-HSP70 antibodies together with AS1 and AS2 molecules, and the data were analyzed separately for the four different reagents. The analysis of AS1 and AS2 binding to different tumor types compared to the expression levels of LOX and HSP70, highlighted that their binding levels were indeed positively correlated with the levels of both biomarkers. Representative images from high to low LOX⁺/HSP70⁺ expression tissues (FIG. 8C, right), with co-staining with AS1 and AS2 confirmed their specificity for those biomarkers (FIG. 8C, left). These results demonstrate the promising potential of our engineering strategy and of AS1/AS2 as inhibitors to be used in various human malignancies.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety

What is claimed is:

1. A method of treating a cancer which is characterized by an up-regulation of expression of lysyl oxidase (LOX) and heat shock protein 70 (HSP70), the method comprising administering to the subject a therapeutically effective amount of a polypeptide comprising a propeptide of lysyl oxidase (LOX), said polypeptide being devoid of LOX catalytic activity, wherein said polypeptide binds to both LOX and heat shock protein 70 (HSP70) with a EC₅₀ of less than 100 nM, thereby treating the cancer.

2. The method of claim 1, wherein said cancer is selected from the group consisting of melanoma, prostate adenocarcinoma, testis embryonal carcinoma, ovarian cancer, uterus carcinoma, pancreatic adenocarcinoma, astrocytoma and glioblastoma.

3. The method of claim 1, wherein said cancer is melanoma.

4. The method of claim 1, wherein said cancer is metastasized.

5. The method of claim 1, further comprising selecting the subject for treatment by analyzing the level of said LOX and said HSP70 in a sample of the subject, wherein when said level is above a predetermined level, the subject is selected as a candidate for treatment.

6. An article of manufacture comprising:
(i) a polypeptide comprising a propeptide of lysyl oxidase (LOX), said polypeptide being devoid of LOX catalytic activity, wherein said polypeptide binds to both LOX and heat shock protein 70 (HSP70) with a EC₅₀ of less than 100 nM; and
(ii) an immune modulating agent.

7. The method of claim 1, wherein said polypeptide comprises the amino acid sequence as set forth in SEQ ID NO: 1.

8. The method of claim 7, wherein said polypeptide is glycosylated on at least one of or at least two of N81, N97 and N144 of SEQ ID NO: 1.

9. The method of claim 7, wherein said polypeptide is glycosylated on N81, N97 and N144 of SEQ ID NO: 1.

10. The method of claim 7, wherein said polypeptide is not glycosylated on N81, N97 and N144 of SEQ ID NO: 1.

11. The method of claim 1, wherein said propeptide of LOX is of human LOX.

12. The method of claim 1, wherein said polypeptide comprises a modification which imparts said polypeptide with enhanced stability under physiological conditions as compared to a native form of said polypeptide not comprising said modification.

13. The method of claim 12, wherein said modification comprises a proteinaceous modification.

14. The method of claim 13, wherein said proteinaceous modification is selected from the group consisting of immunoglobulin, human serum albumin, and transferrin.

15. The method of claim 14, wherein said immunoglobulin comprises an Fc domain.

16. The method of claim 15, wherein said polypeptide comprises the amino acid sequence as set forth in SEQ ID NOs: 12 or 13.

17. The method of claim 1, wherein the polypeptide is a chimeric polypeptide.

18. The method of claim 12, wherein said modification comprises a chemical modification.

19. The method of claim 18, wherein said chemical modification is a polymer.

20. The method of claim 1, further comprising administering to the subject an immune modulating agent.
